(19) United States
(12) Patent Application Publication
(10) Pub. No.: US 2026/0325989 A1
(43) Pub. Date: Oct. 22, 2026

(54) HIGH PERFORMANCE LIQUID CHROMATOGRAPHY EQUIPMENT-BASED AUTOMATION (HPLC-A) SYSTEM FOR AUTOMATED SYNTHESIS

(71) Applicant: Saint Louis University, Saint Louis, MO (US)

(72) Inventors: Alexei V. Demchenko, Glen Carbon, IL (US); Gustavo A. Kashiwagi, Saint Louis, MO (US)

(21) Appl. No.: 19/462,643

(22) Filed: Jan. 28, 2026

Related U.S. Application Data
(60) Provisional application No. 63/753,734, filed on Feb. 4, 2025.

(51) Int. Cl. B01D 15/14; B01D 15/10; B01D 15/24; B01J 19/00; C07H 1/06; C07H 3/04; C07H 15/04; C07H 15/18; C07H 19/067; C07H 19/12; C07J 17/00

(52) U.S. Cl. CPC B01D 15/14; B01D 15/102; B01D 15/247; B01J 19/0013; B01J 19/004; C07H 1/06; C07H 3/04; C07H 15/04; C07H 15/18; C07H 19/067; C07H 19/12; C07J 17/005; B01J 2219/00094

(57) ABSTRACT

The present disclosure relates to systems and methods for synthesizing organic molecules, carbohydrate building blocks, nucleosides, oligosaccharides, and drug-like molecules. In particular, the present disclosure relates to a high performance liquid chromatography equipment-based automation (HPLC-A) system suitable for synthesizing organic molecules, carbohydrate building blocks, nucleosides, oligosaccharides, glycans, and drug-like molecules.

### CROSS-REFERENCE TO RELATED APPLICATIONS

This application claims priority to and benefit of U.S. Provisional Patent Application No. 63/753,734, filed on Feb. 4, 2025, which is incorporated by reference in its entirety.

### STATEMENT OF GOVERNMENT SUPPORT

The invention was made with government support under GM152135 awarded by the National Institutes of Health/National Institute of General Medical Sciences. The government has certain rights in the invention.

### BACKGROUND OF THE DISCLOSURE

The present disclosure relates generally to research and medicine. More particularly, the present disclosure relates to systems and methods for synthesizing organic molecules, carbohydrate building blocks, nucleosides, small and medium size oligosaccharides, glycans, and other drug-like molecules.

Carbohydrates form the basis of all living organisms and are ubiquitous both in nature and in medicine. The rapid growth of research and development in glycoscience demands the development of rapid, efficient, and simple procedures for carbohydrate synthesis. Current methods for the synthesis of glycans are highly sophisticated and operationally complex, leading to a substantial barrier to entry for the technology.

A majority of all syntheses are conducted manually, requiring trained personnel to spend extended working hours on reaction setup and subsequent purification steps. In 2012, Seeberger reported “the first fully automated solid-phase oligosaccharide synthesizer (‘SPOS’).” This synthesizer was then commercialized as Glyconeer 2.1; a few have been installed including 4 in North America. However, the impact of this approach beyond the Seeberger lab remains modest and only one external article highlighting the use of the commercial Glyconeer has been published to date. The Glyconeer is expensive and complex, requiring dedicated personnel. A key advantage of the solid-phase synthesis is the simplified purification of reaction products due to their immobilization on a solid support, an advantage that increases with the number of required purification steps. However, the commercial Glyconeer is not well suited for the production of small molecules, monosaccharides via protecting group manipulations, synthesis of disaccharides and short oligosaccharides. Notably, monitoring coupling efficiency requires an additional cleavage step in which the product is released from the solid support, a requirement that is absent in solution-phase approaches. In addition, solid-phase synthesis (“SPS”) of carbohydrates generally requires a large excess of glycosyl donors compared to reactions in solution phase. In automated SPS approaches, typically 5 to 20 or even more equivalents of glycosyl donors are required per each glycosylation cycle; accordingly, the reduced cost of repetitive purification steps must be balanced against the increased cost associated with excess reactants and reagents. As a result, SPS methods have limited applicability to expensive donors, coupling reactions with known low yields, and syntheses in which a convergent strategy is contemplated.

United States Patent Application Publication No. 2007/0265441 describes an apparatus for the efficient synthesis of oligosaccharides on a solid support. According to the application, oligosaccharides are formed by subunit addition to terminal subunits immobilized on solid-phase particles.

Kashiwagi et al. (Chem Eur J 2024) describes an HPLC-based system that uses a glass syringe and a glass condenser for a reactor and the HPLC quaternary pump system to transfer the reaction mixture. This system used heating tape and thermoregulatory fluid for maintaining temperature. A solution phase reactor enabled reactions where electrophiles and nucleophiles were used in a much lower ratio compared to that used in the SPS approach.

Accordingly, a need exists for chemical methods and automated systems for synthesizing and purifying organic molecules, carbohydrate building blocks, nucleosides, small and medium size oligosaccharides, and other drug-like molecules.

This need for chemical methods and automated systems for synthesizing and purifying organic molecules, carbohydrate building blocks, nucleosides, small and medium size oligosaccharides, and other drug-like molecules is met in the present disclosure with an affordable and accessible automation platform that enables both specialists and non-specialists to perform the synthesis of glycans from renewable precursors. Some automated reactions can be accurately reproduced by a minimally trained researcher using the system and methods of the present disclosure. Automated synthesis offers operational simplicity by delivering all reagents using standard HPLC components and convenient real-time reaction monitoring of every step. Advantageously, the high performance liquid chromatography equipment-based automation (HPLC-A) of the present disclosure represents a highly accessible method for synthesis because many scientists already have easy access to HPLC equipment.

### BRIEF DESCRIPTION OF THE DISCLOSURE

The present disclosure is generally related to systems and methods for synthesizing organic molecules, carbohydrate building blocks, nucleosides, small and medium size oligosaccharides, glycans, and other drug-like molecules.

In one aspect, the present disclosure is directed to a high performance liquid chromatography equipment-based automation (HPLC-A) system for the automated synthesis and purification of organic molecules, carbohydrate building blocks, nucleosides, small and medium size oligosaccharides, glycans, and other drug-like molecules.

In one aspect, the present disclosure is directed to a method for automated synthesis of organic molecules in solution using a batch reactor.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a flow diagram illustrating a prior art system for HPLC-based automated synthesis and purification of carbohydrate building blocks.

FIG. 2 is a flow diagram illustrating an embodiment of a system for HPLC-based automated synthesis and purification of carbohydrate building blocks of the present disclosure.

FIG. 3 depicts upstream and downstream flows, thermoregulating fluid flows, and electrical signal flows according to one embodiment of the present disclosure.

FIG. 4A depicts selected parts of the synthesizer of the embodiment.

FIG. 4B is an illustration depicting a two-way valve plausible connectivity.

FIG. 5 is an illustration depicting an exemplary embodiment of a jacketed reactor.

FIG. 6A depicts an electrical circuit illustrating how selected components are interconnected to enable control of the reaction mixture transfer process by a programmable logic controller (PLC) executing the uploaded logic program. FIG. 6B depicts one example for repurposing an electrical signal managed by HPLC software to a new functionality. In the case of the figure, the signal triggers the operation of a cooling of a reactor.

FIG. 7 depicts a table of logic input-output relationships implemented by the PLC of the claimed embodiment.

FIG. 8 is a schematic illustration of the logic program uploaded (downloaded) to PLC program according to the embodiment.

FIG. 9, according to the embodiment, shows examples of the preparative chromatograms obtained after the automated synthesis of the disaccharide 3, glycolipid 6, disaccharide 8, and galactofuranosyl conjugate 10.

FIG. 10, according to the embodiment, shows examples of the preparative chromatograms obtained after the automated synthesis of the galactopyranosyl conjugate 13, disaccharide 15, and disaccharide 17.

FIG. 11, according to the embodiment, shows examples of the preparative chromatograms obtained after the automated synthesis of the conjugated monosaccharide 20, conjugated trisaccharide 22, and conjugated pentasaccharide 23.

FIG. 12, according to the embodiment, shows examples of the preparative chromatograms obtained after the automated synthesis of the picolinate monosaccharide 24, pentenoylated monosaccharide 25, levulinoylated monosaccharide 26, and Fmoc free thiofuranoside 27.

FIG. 13, according to the embodiment, shows examples of the preparative chromatograms obtained after the automated synthesis of Fmoc free thiopyranoside 29, pyranosyl trichloroacetimidates 31, and furanosyl trichloroacetimidate 33.

FIG. 14, according to the embodiment, shows examples of the preparative chromatograms obtained after the automated synthesis of the nucleosides 35 and 36.

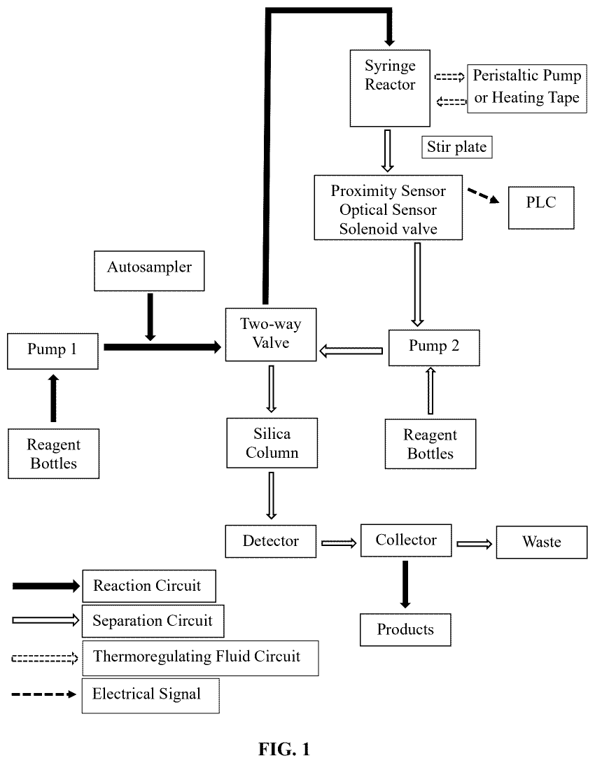

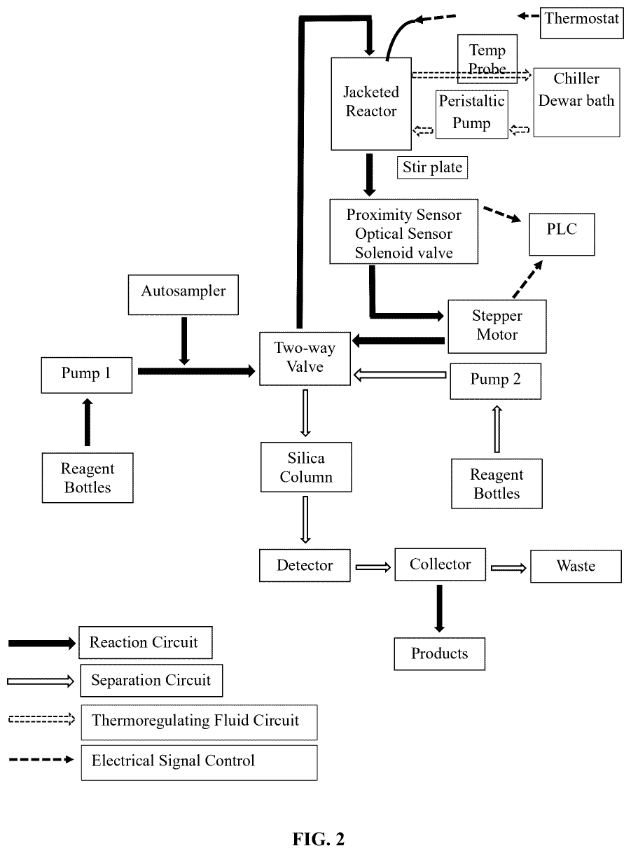

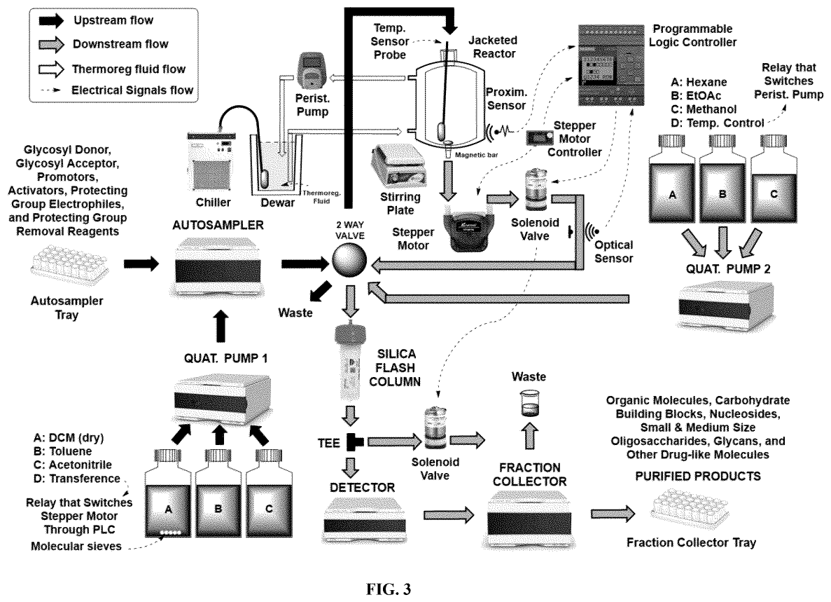

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is directed to systems and methods for synthesizing and purifying organic molecules, carbohydrate building blocks, nucleosides, small and medium size oligosaccharides, glycans, and other drug-like molecules. The system and methods provide rapid, efficient, and simple procedures for synthesis and purification of organic molecules, carbohydrate building blocks, nucleosides, small and medium size oligosaccharides, glycans, and other drug-like molecules. The high performance liquid chromatography equipment-based automation (HPLC-A) system of the present disclosure represents a highly accessible method for synthesis because many scientists already have easy access to HPLC equipment, resulting in a low barrier to entry for the technology. Automated synthesis offers operational simplicity by delivering all reagents using standard HPLC system, wherein the HPLC system also performs separation of the reaction mixture and collects the purified product via its fraction collector module.

In one aspect, the present disclosure is directed to a high performance liquid chromatography equipment-based automation (HPLC-A) system for synthesizing organic molecules, carbohydrate building blocks, nucleosides, small and medium size oligosaccharides, glycans, and other drug-like molecules. The present disclosure describes embodiments of the HPLC-A system in the context of synthesizing a carbohydrate. It should be readily understood by one of ordinary skill in the art that the HPLC-A system and devices can be used to synthesize organic molecules, carbohydrate building blocks, nucleosides, small and medium size oligosaccharides, and other drug-like molecules.

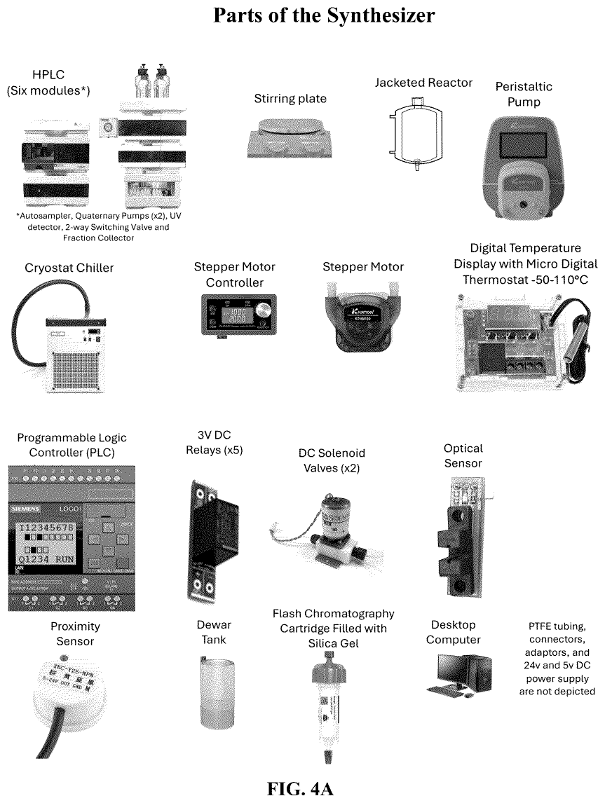

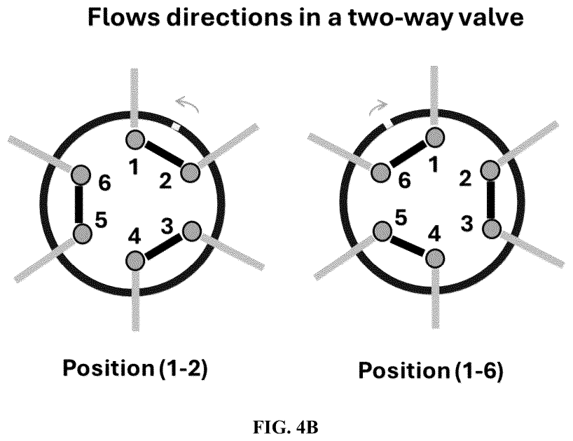

The HPLC-A system includes a high performance liquid chromatography module, a jacketed reactor, a chiller, a chromatography column, pumps, a stirring plate, a logic controller, sensors, valves, a temperature sensor, and a computer. Selected parts of the HPLC-A system are depicted in FIGS. 4A and 4B.

Suitable pumps include peristaltic pumps, controllable stepper motors, and combinations thereof. Suitable peristaltic pumps include high-flow peristaltic pumps, low-flow peristaltic pumps, and combinations thereof. In a particularly suitable embodiment, the HPLC-A system includes two pumps, wherein one peristaltic pump is a high-flow peristaltic pump and the one peristaltic pump is a low-flow peristaltic pump.

Suitable sensors include proximity sensors, optical sensors, and combinations thereof.

Controllable valves include solenoid valves. In a particularly suitable embodiment, the HPLC-A system includes two controllable solenoid valves wherein one controllable solenoid valve is positioned between the reactor and a two-position switch valve, and the other controllable solenoid valve is positioned downstream the column.

The high performance liquid chromatography module includes a chromatography column, an autosampler, a detector, a two-position switch valve, and a fraction collector. Selected instruments that composed the HPLC module are depicted in FIGS. 3, 4A and 4B.

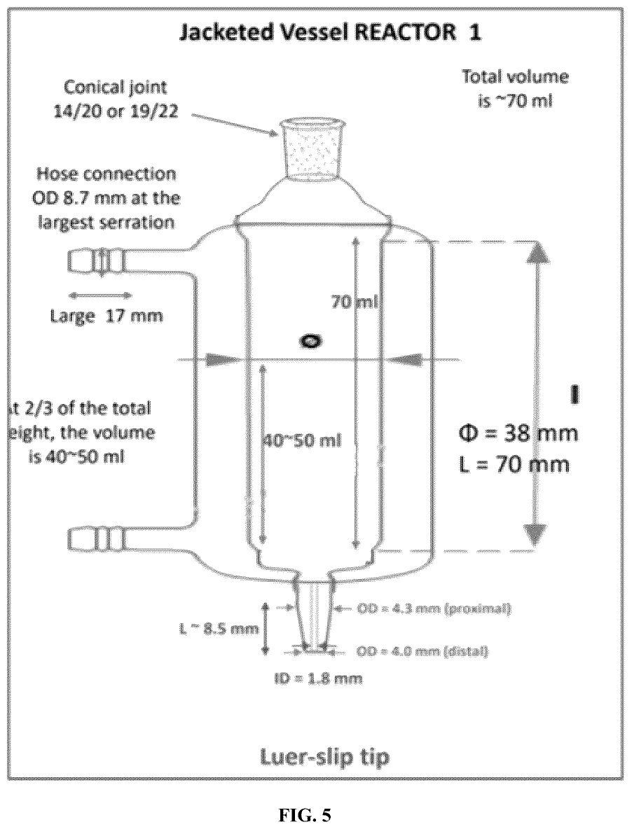

A particularly suitable jacketed reactor is commercially available from Adams & Chittenden Scientific Glass70 mL Jacketed glycan synthesis vessel—14/20F top, 41sw inner, 64sw outer, 301-01 jacket HB, luer joint male 2 mm ID (see, FIG. 5). A suitable luer adapter comprises an Idex threaded adapter having a 0.050-inch bore, a male luer lock, and a female 10-32 coned fitting, constructed from PEEK. Prior to connecting the adapter to the luer joint of the reactor, a small amount of glass wool is loosely packed within the adapter, serving as an inert filter.

A particularly suitable chromatography column is a flash chromatography column. The selection of the size of the flash chromatography cartridge depends on the scale of the reaction along with the chromatographic properties of the reaction products.

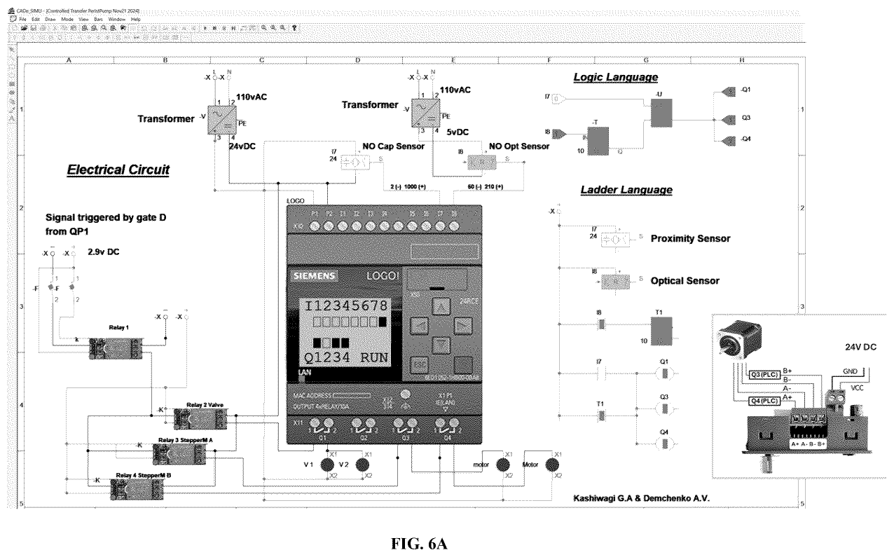

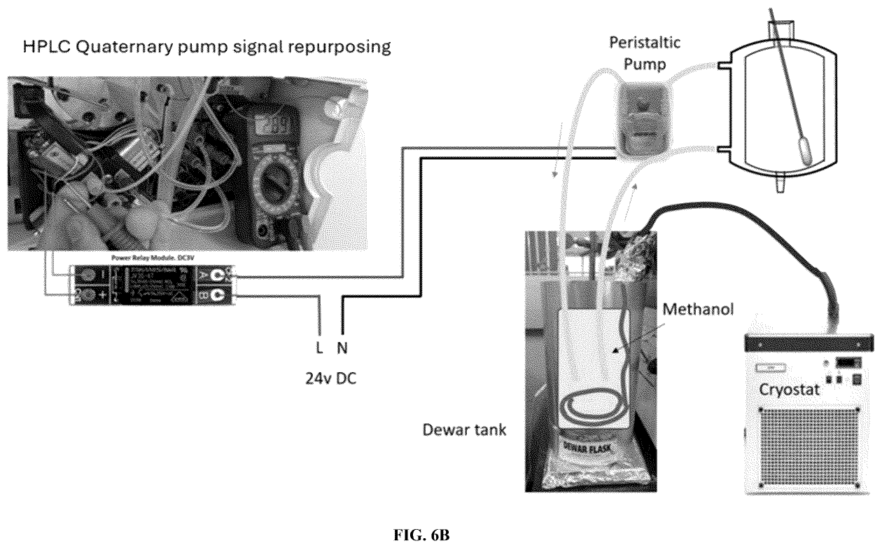

Suitable pumps include peristaltic pumps and quaternary HPLC pumps. A particularly suitable pump is a peristaltic pump equipped with a low pulsation stepper motor and a chemically resistant tube material which is inert to reaction mixture conditions. Use of a peristaltic pump safeguards the quaternary HPLC pump system, which is considerably more expensive than a peristaltic pump. The transfer of the reaction mixture from the jacketed reactor to the chromatographic column is performed by a peristaltic pump that is controlled by a stepper motor controller. The process of transfer is triggered by an electrical signal that comes from HPLC through a programmable logic controller (PLC). The incorporation of a peristaltic pump for reaction transfer in the disclosed invention is an improvement from the setup reported in Kashiwagi et al. (Carbohydr. Res. 2025). The reaction mixture now passes through the peristaltic pump's BPT tube, which safeguards the quaternary pump of the HPLC. A particularly suitable peristaltic pump is the stepper motor Brand: Kamoer KPHM100 Motor STB10. 10 Watts, low pulsation, 0-180 mL flow, 24V DC stepper motor. Long service life high-precision liquid dosing pump 0-180 mL/min 3 mm ID×5 mm OD. A suitable stepper motor controller is a commercially available Stepper Motor Controller NEMA 17/23 DC 5-30V 4A brand Eye.sis, Max Forward/Reverse Angle Pulse Speed Control Module. The flow of the stepper motor can be set by the NEMA controller and be activated or not by the PLC terminals output terminals, as it is depicted in the electrical circuit (FIG. 6A).

The computer of the HPLC-A system includes software configured to control the high performance liquid chromatography module. The particular software program sequence will depend on the synthesis to be performed. Each software program comprises a sequence or a set of commands that control main processes involved in a given synthesis. Such processes include cleaning of upstream tubing with one or more solvents; delivery of solutions containing reactants and reagents; maintaining the reaction mixture for a predetermined duration at a preset temperature; transferring the reaction mixture to a separation section comprising a chromatographic column; separating the product by a preset chromatographic method; and collecting the purified product using a fraction collector module. Generally talking, HPLC software program allows the operator to define the washing solvent and the number of times needed for washing upstream ducts, define number of solutions to be delivered to the reactor, the period of time which reaction can be performed at two different temperatures, the chromatographic solvent and method, the wavelength to detect the presence of product and method of collection of products (collection can be triggered by UV detector signals or by time or by volume).

Because a principal process involves transferring the reaction mixture to the chromatographic column, and because different reactions may involve different reaction volumes, a closed-loop process control system with sensors and solenoid valves, is incorporated in the embodiment, under the control of a programmable logic controller (PLC).

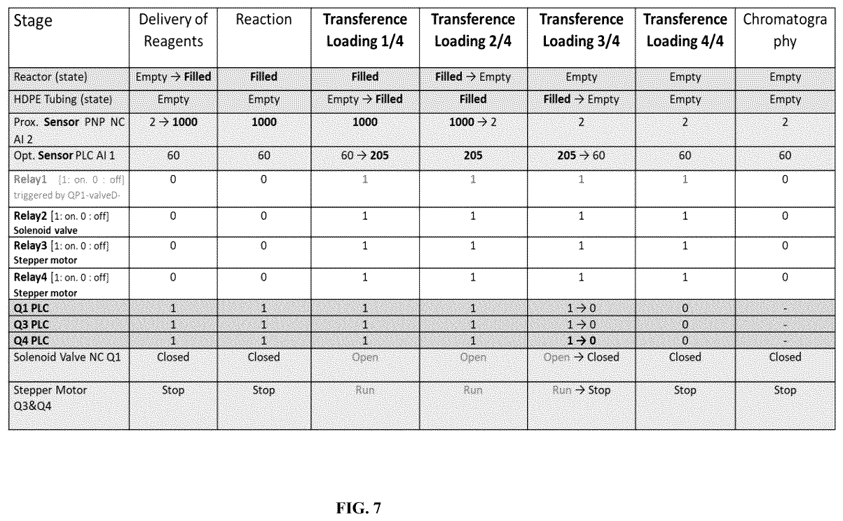

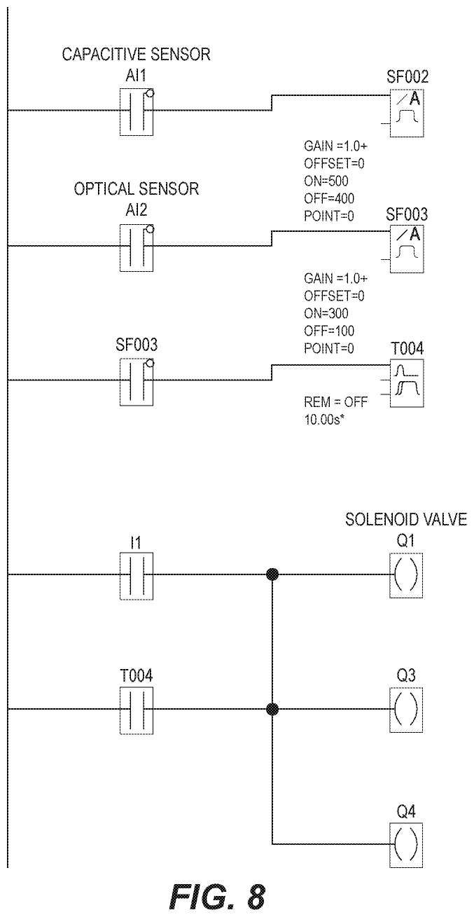

The programmable logic controller (PLC) added to the HPLC-A system includes software configured to control the transfer of the reaction mixture from the reactor to the chromatographic column. The transfer process is triggered by an electrical signal that comes from HPLC throughout a PLC. The particular software program sequence will depend on the synthesis to be performed. An example of the program that led to a controlled transfer is depicted by in FIG. 8. The program depicted follows a table of logic input-output that represent the behavior of the solenoid valves at each stage of the automation process (FIG. 7).

The HPLC-A system can further include a thermocontrol pump. A particularly suitable thermocontrol pump is a peristaltic pump for thermocontrol that circulates a “thermoregulatory fluid” that conditions the temperature of the reaction. It can be attached to a chiller or a heater, allowing the jacketed reactor to reach temperatures ranging from −50 °C to +80 °C. In the present disclosure, the peristaltic pump can circulate thermoregulatory fluid with a higher flow rate to enable reaching lower temperatures. Temperature control is further enhanced by implementing a low temperature resistant and flexible BPT tubing. A particularly suitable peristaltic pump for thermocontrol function is Kamoer KCP-D 24V DC, 24W. Small lab liquid dosing pump 3 mm ID×5 mm OD. Flowrate 50-100 mL/min. Suitable commercially available pump tube material is Pharmed BPT tube having a working temperature range-51 °C to ~132 °C.

A particularly suitable chiller is a Cryostat chiller EK90.

The HPLC-A system further includes an immersible temperature probe system that allows for real time in line temperature measurement. A suitable commercially available immersible temperature probe system is a Pcs NTC 10K 3950 Temperature Probe (−25 °C to 125 °C. Temperature Sensor Probe Stainless Steel Housing Thermistor Temp Sensor—3 Meters/9.8 Ft Cable Length. The temperature sensor is plugged to 12V DC Digital Temperature Controller Board Micro Digital Thermostat −50 °C-110 °C. Electronic Temperature Temp Control Module Switch with 10A one-channel relay and is waterproof. The immersible temperature probe system allows for real time in line temperature measurement of the reaction mixture and better control and monitoring of temperature.

The HPLC-A system further includes a separation chromatography column. A particularly suitable separation chromatography column for automated synthesis of glycans or protecting group manipulations is a normal phase silica column. A particularly suitable column is the Biotage Sfar Silica HC D flash chromatography cartridge. Sizes can vary depending on the reaction scale and chromatography profile of the reaction products. The cartridge can be recycled several times and repacked with fresh silica. A particularly suitable normal phase silica is Silicycle Siliaflash, 60 Å, particle size 20-45 μm.

Along with the main components of the HPLC-A system, minor components include magnetic bars, PEEK adapters and HDPE lines, HPLC PTFE tubing (0.7 mm id), tee connectors, unions, power cables, relays, septa, balloon, molecular sieves and an air moisture trap.

The HPLC-A system further includes the use of two commercial software packages concomitantly: the software compatible with the HPLC (for example, Chemstation for an Agilent HPLC) and the software compatible with the PLC (LOGO!Soft Comfort for a Siemens PLC). Once the logic program is uploaded to PLC, it is not necessary to open it while the synthesis is in progress.

In another aspect, the present disclosure is directed to an automated method for synthesizing a product in solution phase, under batch conditions, using the HPLC-A system. In the claimed embodiment, the HPLC-A system operates using a batch reactor in a solution-phase configuration. Such an arrangement allows for reactions to be initiated under selected starting conditions and may help to reduce consumption of reactants and reagents relative to certain alternative approaches like automated SPS approaches. Operation of the system is conducted by implementing the HPLC control software, which can facilitate programming and operation of the system, and may lower the barrier of entry to this technology, consequently, enhancing the accessibility. The HPLC-A system is suitable to synthesize and purify products selected from organic molecules, carbohydrate building blocks, nucleosides, oligosaccharides, and drug-like molecules.

For the automated method for synthesis, the HPLC-A system is loaded with vials containing reagent and reactant solutions, sometimes using dried solvents. Also, the HPLC bottles of the system need to be filled with proper solvents (needed for reactions, for cleaning lines, and for chromatography process). After loading, the user downloads the appropriate programs associated with the specific synthesis and separation. The user needs to turn on the peripherical components (chiller, peristaltic pumps, stirring plate, sensors, solenoid valves, and PLC) associated with the temperature control, mixing, and transfer. Then the user initiates the automation processes (or sequence) by selecting the “run sequence” option. When the automation sequence ends, the user opens the fraction collector door and selects the product collected from the tray. The user can judge which tubes to collect based on the chromatogram reported by the HPLC software.

### EXAMPLES

Examples 1 to 10 describe performance of the of the fully automated HPLC-A system when a glycosylation is performed using a specific promoter (specifically one called BDMPH which is described below) and specific temperature conditions. Results were compared with respective syntheses performed manually. Examples 11 to 15 describe performance of the fully automated HPLC-A system when protecting group manipulations are performed under certain conditions. Examples 16 and 17 describe the fully automated synthesis of common trichloroacetimidate glycosyl donors. Examples 18 and 19 describe the fully automated synthesis of two nucleosides

### Example 1

This Example describes 1-bromo-3,5-dimethyl-5-phenylhydantoin (BDMPH)/tert-butyldimethylsilyl trifluoromethanesulfonate (TBDMSOTf) promoted glycosylations. BDMPH was synthesized by brominating a known compound, 3,5-dimethyl-5-phenylhydantoin (Shintani et al. Chem Pharm Bull 2021). 1.3 g of sodium hydroxide was added to the stirred suspension of 5.6 g of 3,5-dimethyl-5-phenylhydantoin in 24 mL of H₂O. This mixture was cooled to 0 °C then 2.1 mL of bromine was added to this mixture. The mixture was allowed to warm back to room temperature for over one hour. The resultant orange suspension was filtered with a Büchner funnel, and the solids was rinsed with H₂O until it appeared as beige solids. The solids were further dried on high vacuum, protected from light. The dried solids were purified by recrystallization from 50 mL of ethyl acetate heating at 75 °C. The crystals were filtered with a Büchner funnel and rinsed with hexanes. The crystals were further dried on high vacuum, protected from light to yield 5.4 g (69%) of BDMPH. ¹H NMR (600 MHz, CDCl₃): δ 7.44-7.41 (m, 2H, ArH), 7.40-7.37 (m, 1H, ArH), 7.31-7.29 (m, 2H, ArH), 2.89 (s, 3H, NCH₃), 1.87 (s, 3H, CH₃); ¹³C NMR (150 MHz, CDCl₃): δ 172.4 (C═O), 153.0 (C═O), 135.4 (ArC), 129.3 (ArC), 129.2 (ArC), 125.9 (ArC), 68.8 (CPh), 26.5 (CH₃), 20.7 (CH₃); HRMS-ESI-TOF (m/z): [M+Na]⁺ calcd for C₁₁H₁₁BrN₂NaO₂, 304.9902; found, 304.9899.

The compounds were detected by examination under UV light and by charring with 10% sulfuric acid in methanol. Column chromatography was performed on silica gel 60 (70-230 mesh), and reactions were monitored by TLC on Kieselgel 60 F254. Solvents were removed under reduced pressure at <40 °C. CH₂Cl₂ (DCM) and ClCH₂CH₂Cl (1,2-DCE) were distilled from CaH₂ directly prior to application. Anhydrous DMF was used as it is. Molecular sieves (4 Å), used for reactions, were crushed and activated in vacuo at 390 °C during 8 h in the first instance and then for 2-3 h at 390 °C directly prior to application. Optical rotations were measured using a Jasco polarimeter. ¹H NMR spectra were recorded in CDCl₃ at 400, 600, or 700 MHz. ¹³C NMR spectra were recorded in CDCl₃ at 101, 150, or 176 MHz. The ¹H NMR chemical shifts are given relative using internal tetramethylsilane (TMS, δ_{H}=0 ppm) standard or residual solvent (CHCl₃, δ_{H}=7.26 ppm) for solutions in CDCl₃ and HDO signal (δ_{HOD}=4.8 ppm) in case of samples prepared in D₂O. The ¹³C NMR chemical shifts are referenced to the central signal of CDCl₃ (δ_{C}=77.00 ppm) for solutions in CDCl₃. Compound purity or compound ratios were accessed or calculated by comparing the integration intensities of the relevant signals in their ¹H NMR spectra. Accurate mass spectrometry determinations (HRMS) were performed using Agilent 6230 LCMS ESI TOF mass spectrometer.

This Example describes manual and fully automated syntheses of disaccharide methyl 2,3,5,6-tetra-O-benzoyl-β-D-galactopyranosyl-(1->6)-2,3,4-tri-O-benzyl-α-D-glucopyranoside (3) promoted by BDMPH/TBDMSOTf.

Manual synthesis. The following four solutions were prepared separately using dry dichloromethane at room temperature. 10.2 mg of donor 1 in 0.5 mL, 10.1 mg of acceptor 2 in 0.5 mL, 8.2 mg of BDMPH in 0.5 mL, and 1.7 μL of TBDMSOTf in 0.5 mL. In a 10 mL round bottom flask at room temperature with a solution of donor, acceptor and BDMPH solutions were added. After that, the resultant mixture was cooled to −20 °C and TBDMSOTf solution was added slowly. The reaction mixture was stirred under argon for 0.5 h at −20 °C, when a TLC showed complete consumption of the donor. After that, the whole reaction was loaded into the chromatographic column, which was previously assembled in hexane. 11.1 mg (73%) of pure disaccharide 3 was isolated. Analytical data coincided with the automated procedure described below.

Automation sequence. All reagent solutions were prepared the same day of the experiment and loaded into the autosampler tray along with Toluene. The reactor was previously cleaned with acetone and dried with hot air.

The following four solutions were prepared separately using dry dichloromethane at room temperature. 86.9 mg of donor 1 in 0.9 mL, 86.1 mg of acceptor 2 in 0.9 mL, 70.0 mg of BDMPH in 0.9 mL, and 14 μL of TBDMSOTf in 0.9 mL. The execution of the sequence programmed for this reaction gave a set of fractions collected from the fraction collector. Once evaporated, combined fractions yielded 88.7 mg (72%) of product 3 in pure form. R_{f}=0.70 (hexane/ethyl acetate 2/1, v/v). ¹H NMR (400 MHz, CDCl₃): δ 8.00-7.80 (m, 8H, aromatic), 7.54-7.27 (m, 25H, aromatic), 7.25-7.20 (m, 2H, aromatic), 5.88 (dd, 1H, J_{3′,4′}=J_{3′,4′}=9.7 Hz, H-3′), 5.66 (dd, 1H, J_{3′,4′}=J_{4′,5′}=9.7 Hz, H-4′), 5.58 (dd, 1H, J_{2′,3′}=9.7, J_{1′,2′}=7.8 Hz, H-2′), 4.88 (d, 1H, ²J=11.0 Hz, CHPh), 4.81 (d, 1H, J_{1′,2′}=7.8 Hz, H-1′), 4.72 (d, 1H, ²J=12.1 Hz, CHPh), 4.68 (d, 1H, ²J=11.0 Hz, CHPh), 4.58 (d, 1H, ²J=12.1 Hz, CHPh), 4.60 (dd, 1H, J_{6a′,6b′}=12.1, J_{5′,6a}′=3.5 Hz, H-6a′), 4.53-4.48, (m, 3H, H-1, H-6b′, CHPh), 4.28 (d, 1H, ²J=11.1 Hz, CHPh), 4.14 (m, 1H, H-6a′), 4.09 (m, 1H, H-5′), 3.87 (dd, 1H, J_{2,3}=J_{3,4}=9.3 Hz, H-3), 3.75-3.72 (m, 2H, H-5, H-6b), 3.42 (dd, 1H, J_{2,3}=9.3, J_{1,2}=3.5 Hz, H-2), 3.37 (t, 1H, J_{3,4}=9.3, J_{4,5}=9.3 Hz, H-4), 3.20 (s, 3H, OCH₃) ppm. HRMS for [M+Na]⁺: calcd 1065.3673; found: 1065.3657. The described analytical data coincided with those previously described (Kashiwagi et al. Chem Eur J 2024).

### Example 2

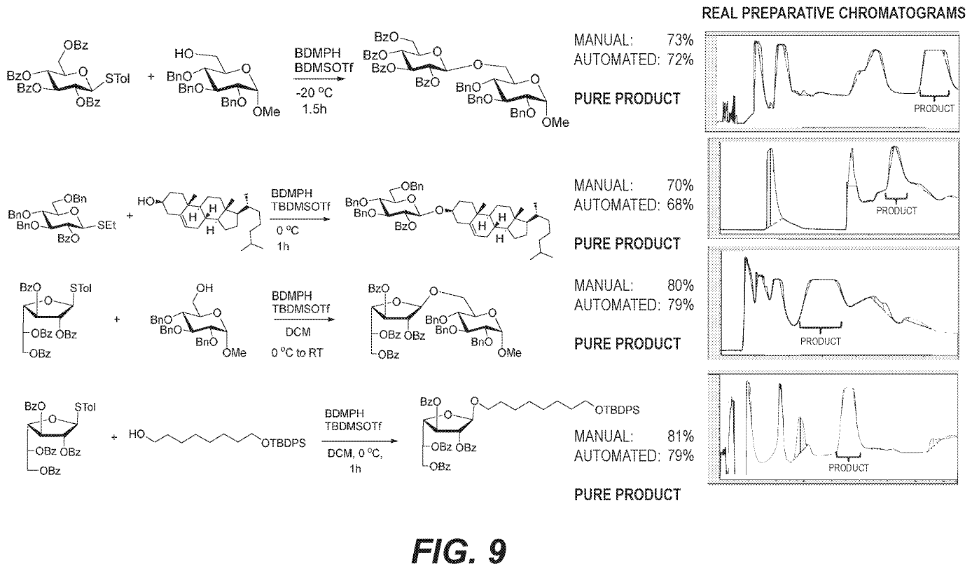

This Example describes manual and fully automated synthesis of cholesteryl(2-O-benzoyl-3,4,6-tri-O-benzyl)-β-D-glucopyranoside (6) synthesis promoted by BDMPH/TBDMSOTf (FIG. 9).

Manual. The following four solutions were prepared separately using dry dichloromethane at room temperature. 10 mg of donor 4 in 0.5 mL, 12.9 mg of acceptor 5 in 0.5 mL, 9.5 mg of BDMPH in 0.5 mL, and 2.0 μL of TBDMSOTf in 0.5 mL. In a 10 mL round bottom flask at room temperature, solution of donor, acceptor and BDMPH were added. After that, the resultant mixture was cooled to 0 °C and TBDMSOTf solution was added slowly. The reaction mixture was allowed to warm to 15 °C and stirred under argon for 25 min, when a TLC showed complete consumption of the donor. After that, the whole reaction was loaded into the chromatographic column, which was previously assembled in hexane. 10.6 mg (70%) of glycolipid 6 was isolated was isolated. Analytical data coincided with the automated procedure described below.

Automation sequence. All reagent solutions were prepared the same day of the experiment and loaded into the autosampler tray along with Toluene. The reactor was previously cleaned with acetone and dried with hot air. The following four solutions were prepared separately using dry dichloromethane at room temperature. 70 mg of donor 4 in 0.9 mL, 90 mg of acceptor 5 in 0.9 mL, 83 mg of BDMPH in 0.9 mL, and 14 μL of TBDMSOTf in 0.9 mL. The execution of the sequence programmed for this reaction gave a set of fractions collected from the fraction collector. Once evaporated, combined fractions yielded 74.4 mg (68%) of product 6 in pure form. R_{f}=0.75 (hexane/ethyl acetate 2:1, v/v). ¹H NMR (400 MHz, CDCl₃): δ 8.02-8.00 (d, 2H, J=7.2 Hz, aromatic), 7.58-7.54 (m, 1H, aromatic), 7.45-7.42 (m, 2H, aromatic), 7.36-7.28 (m, 8H, aromatic), 7.21-7.19 (m, 2H, aromatic), 7.15-7.11 (s, 5H, aromatic), 5.23 (dd, 1H, J=9.4, 8.0 Hz, H-2), 5.21-5.20 (m, 1H, cholesterol), 4.82 (d, 1H, ²J=10.9 Hz, CHPh), 4.72 (d, 1H, ²J=11.1 Hz, CHPh), 4.65 (d, 1H, ²J=11.2 Hz, CHPh), 4.65 (d, 1H, ²J=12.3 Hz, CHPh), 4.62 (d, 1H, J=8.5 Hz, H-1), 4.58 (d, 1H, ²J=11.2 Hz, CHPh) 4.57 (d, 1H, ²J=12.3 Hz, CHPh), 3.83-3.68 (m, 4H, H-3, H-4, H-6a, H-6b), 3.55 (m, 1H, H-5), 3.49-3.45 (m, 1H, cholesterol), 2.15-1.78 (m, 8H, cholesterol), 1.61-0.85 (m, 47H, cholesterol), 0.64 (s, 3H, cholesterol). HRMS for [M+Na]⁺: calcd 945.5645; found: 945.5609. The described data coincided with those previously described (Kashiwagi et al. Chem Eur J 2024).

### Example 3

This Example describes the manual and fully automated synthesis of methyl 2,3,5,6-tetra-O-benzoyl-β-D-galactofuranosyl-(1→6)-2,3,4-tri-O-benzyl-α-D-glucopyranoside (8) synthesis (FIG. 9).

Manual. The following four solutions were prepared separately using dry dichloromethane at room temperature. 49.6 mg of donor 7 in 0.9 mL, 26.2 mg of acceptor 2 in 0.9 mL, 28 mg of BDMPH in 0.9 mL, and 1.7 μL of TBDMSOTf in 0.9 mL. In a 10 mL round bottom flask at room temperature, solution of donor, acceptor and BDMPH were added. After that, the resultant mixture was cooled to 0 °C and TBDMSOTf solution was added slowly. The reaction mixture was stirred under argon for 1 h at 0 °C, when a TLC showed complete consumption of the donor. After that, the whole reaction was loaded into the chromatographic column, which was previously assembled in hexane. 47.1 mg (80%) of pure disaccharide 8 was isolated. Analytical data coincided with the automated procedure described below.

Automation Sequence. All reagent solutions were prepared the same day of the experiment and loaded into the autosampler tray along with Toluene. The reactor was previously cleaned with acetone and dried with hot air. The following four solutions were prepared separately using dry dichloromethane at room temperature. 94.5 mg of donor 7 in 0.9 mL, 50.0 mg of acceptor 2 in 0.9 mL, 38.1 mg of BDMPH in 0.9 mL, and 5.0 μL of TBDMSOTf in 0.9 mL. The execution of the sequence programmed for this reaction gave a set of fractions collected from the fraction collector. Once evaporated, combined fractions yielded 73.2 mg (79%) of product 8 in pure form. R_{f}=0.55 (hexane/ethyl acetate 5:2, v/v). [α]_{D}²⁴+71.9 (c=1, CHCl₃); ¹H NMR (400 MHz, CDCl₃): δ 8.07-8.01 (d, 4H, aromatic), 7.98-7.95 (m, 2H, aromatic), 7.87-7.85 (m, 2H, aromatic), 7.51-7.47 (m, 4H, aromatic), 7.37-7.24 (m, 23H, aromatic), 6.07 (m, 1H, J_{4′,5′}=J^{5′,6a′}=5.7, J_{5′,6b′}=3.4 Hz, H-5′), 5.61 (d, 1H, J_{3′,4′}=5.0 Hz, H-3′), 5.46 (d, 1H, J_{2′,3′}=1.2 Hz, H-2′), 5.29 (s, 1H, H-1′), 4.90 (d, 1H, ²J=11.1 Hz, CHPh), 4.82 (d, 1H, ²J=10.8 Hz, CHPh), 4.72-4.65 (m, 3H, H-4′, H-6a′, H-6b′), 4.71 (d, 1H, ²J=12.1 Hz, CHPh), 4.66 (d, 1H, ²J=11.1 Hz, CHPh), 4.57 (d, 1H, ²J=12.1 Hz, CHPh), 4.57 (d, 1H, J_{1,2}=3.6 Hz, H-1), 4.00 (dd, 1H, J_{2,3}=J_{3,4}=9.2 Hz, H-3), 3.96 (dd, 1H, J_{6a,6b}=11.0, J_{5,6a}=1.8 Hz, H-6a), 3.79 (m, 1H, H-5), 3.75 (dd, 1H, J_{6a,6b}=11.0, J_{5,6b}=4.6 Hz, H-6b), 3.61 (dd, 1H, J_{3,4}=J_{4,5}=9.2 Hz, H-4), 3.45 (dd, 1H, J_{2,3}=9.2, J_{1,2}=3.6 Hz, H-2), 3.34 (s, 3H, OCH₃) ppm; ¹³C NMR (101 MHz, CDCl₃): δ 166.1, 165.7, 165.6, 165.3 (4 C, COPh), 138.8, 138.2, 138.1, 133.4, 133.3, 133.2, 133.0, 129.93, 129.87, 129.8, 129.7, 129.6, 129.5, 129.0, 128.9, 128.44, 128.41, 128.37, 128.34, 128.00, 127.98, 127.9, 127.8, 127.7, 127.6, 105.6 (C-1′), 97.9 (C-1), 82.0 (C-3), 81.7 (C-2′), 81.5 (C-4′), 80.2 (C-2), 77.5 (C-4), 77.4 (C-3′), 75.7 (CH₂Ph), 75.0 (CH₂Ph), 73.3 (CH₂Ph), 70.3 (C-5′), 69.9 (C-5), 65.9 (C-6), 63.8 (C-6′), 55.1 (OCH₃) ppm. HRMS for [M+Na]⁺: calcd 1065.3673; found: 1065.3682.

### Example 4

This Example describes the manual and fully automated synthesis of 8-(tert-butyldiphenylsilyloxy)-oct-1-yl 2,3,5,6-tetra-O-benzoyl-β-D-galactofuranoside (10) (FIG. 9).

Manual. The following four solutions were prepared separately using dry dichloromethane at room temperature. 51 mg of donor 7 in 0.9 mL, 20 mg of acceptor 9 in 0.9 mL, 20.6 mg of BDMPH in 0.9 mL, and 1.8 μL of TBDMSOTf in 0.9 mL. In a 10 mL round bottom flask at room temperature, solution of donor, acceptor and BDMPH were added. After that, the resultant mixture was cooled to 0 °C and TBDMSOTf solution was added slowly. The reaction mixture was stirred under argon for 1 h at 0 °C and the whole reaction was loaded into the chromatographic column. 40.4 mg (81%) of pure title compound 10 was isolated. Analytical data coincided with the automated procedure described below.

Automation sequence. All reagent solutions were prepared the same day of the experiment and loaded into the autosampler tray along with Toluene. The reactor was previously cleaned with acetone and dried with hot air. The following four solutions were prepared separately using dry dichloromethane at room temperature. 95 mg of donor 7 in 0.9 mL, 40.0 mg of acceptor 9 in 0.9 mL, 38.2 mg of BDMPH in 0.9 mL, and 3.6 μL of TBDMSOTf in 0.9 mL. The execution of the sequence programmed for this reaction gave a set of fractions collected from the fraction collector. Once evaporated, combined fractions yielded 78.8 mg (79%) of product 10 in pure form. R_{f}=0.48 (hexane/ethyl acetate 5:1, v/v). [α]_{D}²⁵−5.9 (c=1, CHCl₃). ¹H NMR (400 MHz, CDCl₃): δ 8.09-8.03 (d, 4H, aromatic), 7.98-7.96 (m, 2H, aromatic), 7.92-7.89 (m, 2H, aromatic), 7.68-7.66 (m, 4H, aromatic), 7.55-7.48 (m, 4H, aromatic), 7.43-7.26 (m, 14H, aromatic), 6.07 (m, 1H, J_{5,6b}=6.9, J_{5,6a}=4.7, J_{4,5}=3.5 Hz, H-5), 5.63 (d, 1H, J_{3,4}=5.2 Hz, H-3), 5.46 (d, 1H, J_{2,3}=1.1 Hz, H-2), 5.30 (s, 1H, H-1), 4.79 (d, 1H, J_{6a,6b}=11.8, J_{5,6a}=4.7 Hz, H-6a), 4.74 (d, 1H, J_{6a,6b}=11.8, J_{5,6b}=6.9 Hz, H-6b), 4.64 (dd, 1H, J_{3,4}=5.2, J_{4,5}=3.5 Hz, H-4), 3.74 (m, 1H, SiOCH₂R), 3.64 (m, 2H, OCH₂CH₂R), 3.53 (m, 1H, SiOCH^{b}R), 1.63-1.60 (m, 2H), 1.55-1.50 (m, 2H), 1.35-1.24 (m, 8H), 1.05 (s, 9H, ^{t}Bu) ppm; ¹³C NMR (101 MHz, CDCl₃): δ 166.1, 165.7, 165.6, 165.4 (4C, COPh), 135.6, 134.2, 130.0, 129.9, 129.8, 129.7, 129.5, 128.37, 128.36, 128.32, 127.5, 105.6 (C-1), 82.1 (C-2), 81.2 (C-4), 77.6 (C-3), 70.3 (C-5), 70.3 (C-5), 67.7 (SiOCH₂R), 64.0 (ClOCH₂R), 63.5 (C-6), 32.6, 29.5, 29.4, 29.3, 26.9 (C(CH₃)₃), 26.1, 25.7, 19.2 ppm; HRMS for [M+Na]⁺: calcd 985.3959; found: 985.3917.

### Example 5

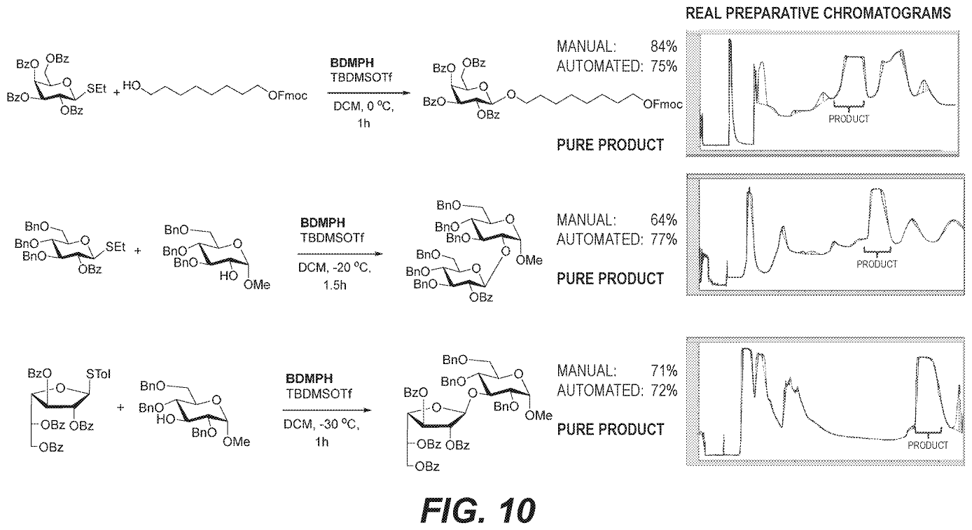

This Example describes the manual and fully automated synthesis of 8-[O-(9-fluorenylmethyloxycarbonyl)]-oct-1-yl 2,3,4,6-tetra-O-benzoyl-β-D-galactopyranoside (13) synthesis (FIG. 10).

Manual. The following four solutions were prepared separately using dry dichloromethane at room temperature. 52.1 mg of donor 11 in 0.9 mL, 25 mg of acceptor 12 in 0.9 mL, 23 mg of BDMPH in 0.9 mL, and 3.0 μL of TBDMSOTf in 0.9 mL. In a 10 mL round bottom flask at room temperature, solution of donor, acceptor and BDMPH were added. After that, the resultant mixture was cooled to 0 °C and TBDMSOTf solution was added slowly. The reaction mixture was stirred under argon for 1 h at 0 °C and the whole reaction was loaded into the chromatographic column. 39.8 mg (84%) of pure title compound 13 was isolated. Analytical data coincided with the automated procedure described below.

Automated sequence. All reagent solutions were prepared the same day of the experiment and loaded into the autosampler tray along with Toluene. The reactor was previously cleaned with acetone and dried with hot air. The following four solutions were prepared separately using dry dichloromethane at room temperature. 60.6 mg of donor 11 in 0.9 mL, 25.0 mg of acceptor 12 in 0.9 mL, 26.9 mg of BDMPH in 0.9 mL, and 6.2 μL of TBDMSOTf in 0.9 mL. The execution of the sequence programmed for this reaction gave a set of fractions collected from the fraction collector. Once evaporated, combined fractions yielded 48 mg (75%) of product 13 in pure form. R_{f}=0.45 (hexane/ethyl acetate 3:1, v/v). [α]_{D}²⁵+71.7 (c=1, CHCl₃). ¹H NMR (400 MHz, CDCl₃): δ 8.11-8.09 (d, 2H, aromatic), 8.03-7.98 (m, 2H, aromatic), 7.98-7.96 (m, 2H, aromatic), 7.80-7.75 (m, 4H, aromatic), 7.63-7.26 (m, 16H, aromatic), 7.26-7.22 (m, 2H, aromatic), 6.00 (dd, 1H, J_{3,4}=3.5, J_{4,5}=1.2 Hz, H-4), 5.80 (dd, 1H, J_{2,3}=10.4, J_{1,2}=7.9 Hz, H-2), 5.61 (dd, 1H, J_{2,3}=10.4, J_{3,4}=3.5 Hz, H-3), 4.80 (d, 1H, J_{1,2}=7.9 Hz, H-1), 4.70 (d, 1H, J_{6a,6b}=11.3, J_{5,6a}=6.7 Hz, H-6a), 4.42 (dd, 1H, J_{6a,6b}=11.8, J_{5,6b}=6.7 Hz, H6b), 4.41 (d, 1H, 3J-7.4 Hz, CH₂(Fmoc)), 4.32 (m, 1H, J_{5,6a}=J_{5,6b}=6.7 Hz, J_{4,5}=1.2 Hz, H-3), 4.26 (t, 1H, 3J=7.4 Hz, CH(Fmoc)), 4.11 (t, 2H, J=6.8 Hz, RCH₂OCO), 3.99 (m, 1H, C1-OCH^{a}R), 3.56 (ddd, 1H, C1-OCH R), 1.62-1.52 (m, 4H), 1.26-1.06 (m, 8H) ppm; ¹³C NMR (101 MHz, CDCl₃): δ 166.0, 165.57, 165.56, 165.2 (4C, COPh), 155.3 (OCOCH₂), 143.5, 141.3, 133.5, 133.3, 133.2, 133.18, 130.0, 129.8, 129.7, 129.4, 129.0, 128.8, 128.6, 128.4, 128.33, 128.25, 127.8, 127.1, 125.1, 120.0, 101.8 (C-1), 71.7 (C-3), 71.2 (C-5), 70.5 (C1-OCH₂R), 69.8 (C-2), 69.6 (CH₂(Fmoc)), 68.3 (RCH₂OCO), 68.1 (C-4), 62.0 (C-6), 46.8 (CH(Fmoc)), 29.3, 29.1, 29.0, 28.6 25.7, 25.5 ppm; HRMS for [M+Na]⁺: calcd 969.3462; found: 969.3518.

### Example 6

This Example describes the manual and fully automated synthesis of methyl 2-O-(2-O-benzoyl-3,4,6-tri-O-benzyl-β-D-glucopyranosyl)-3,4,6-tri-O-benzyl-α-D-glucopyranoside (15) synthesis (FIG. 10).

Manual. The following four solutions were prepared separately using dry dichloromethane at room temperature. 38.1 mg of donor 7 (Forsythe, N) (1.0 eq.) in 0.9 mL, 40 mg of acceptor 14 (1.35 eq.) in 0.9 mL, 19.8 mg of BDMPH (1.1 eq.) in 0.9 mL, and 6.0 μL of TBDMSOTf (0.4 eq.) in 0.9 mL. In a 10 mL round bottom flask at room temperature, solution of donor, acceptor and BDMPH were added. After that, the resultant mixture was cooled to −20 °C and TBDMSOTf solution was added slowly. The reaction mixture was stirred under argon for 1 h at −20 °C and the whole reaction was loaded into the chromatographic column. 60 mg (70%) of pure title compound 15 was isolated. Analytical data coincided with the automated procedure described below.

Automated sequence. All reagent solutions were prepared the same day of the experiment and loaded into the autosampler tray along with toluene. The reactor was previously cleaned with acetone and dried with hot air. The following four solutions were prepared separately using dry dichloromethane at room temperature. 52.5 mg of donor 7 (1.0 eq) in 0.9 mL, 55.0 mg of acceptor 14 (1.35 eq.) in 0.9 mL, 62 mg of BDMPH (2.5 eq) in 0.9 mL, and 8.0 μL of TBDMSOTf (0.4 eq) in 0.9 mL. The execution of the sequence programmed for this reaction gave a set of fractions collected from the fraction collector. Once evaporated, combined fractions yielded 67 mg (77%) of product 15 in pure form. R_{f}=0.50 (hexane/ethyl acetate 3:1, v/v). [α]_{D}²⁵+56.9 (c=1, CHCl₃). ¹H NMR (400 MHz, CDCl₃): δ 7.84-7.81 (d, 2H, aromatic), 7.34-7.25 (m, 14H, aromatic), 7.20-7.06 (m, 15H, aromatic), 6.98-7.94 (m, 4H, aromatic), 5.42 (dd, 1H, J_{2′,3′}=8.0, J_{1,2′}=8.0 Hz, H-2′), 5.02 (d, 1H, J_{1,2}=3.4 Hz, H-1), 4.85 (d, 1H, J_{1,2′}=8.0 Hz, H-1′), 4.79 (d, 1H, ²J=10.8 Hz, CHPh), 4.70 (d, 1H, ²J=11.1 Hz, CHPh), 4.62-4.58 (m, 6H, 3×CH₂Ph), 4.52 (d, 1H, ²J=12.0 Hz, CHPh), 4.47 (d, 1H, ²J=11.6 Hz, CHPh), 4.44 (d, 1H, ²J=10.7 Hz, CHPh), 4.34 (d, 1H, ²J=10.8 Hz, CHPh), 3.88 (dd, 1H, J_{2,3}=9.7, J_{3,4}=8.8 Hz, H-3), 3.82-3.78 (m, 2H, H-3′, H-4′), 3.75-3.68 (m, 5H, H-2, H-5, H-6a, H-6a′, H-6b), 3.63 (dd, 1H, J=10.4, 1.9 Hz, H-6b′), 3.58 (dd, 1H, J_{4,5}=J_{3,4}=8.7 Hz, H-4), 3.58-3.54 (m, 1H, H-5′), 3.36 (s, 3H, OCH₃), ppm; ¹³C NMR (101 MHz, CDCl₃): δ 165.0 (COPh), 138.7, 138.2, 138.0, 137.9, 137.8, 137.6, 132.8, 129.7, 129.6, 128.4, 128.3, 128.2, 128.18, 128.15, 128.10, 128.02, 127.97, 127.92, 127.87, 127.85, 127.78, 127.72, 127.68, 127.63, 127.58, 127.45, 127.1, 126.96, 102.3 (C-1′), 99.6 (C-1), 83.1 (C-3′), 81.2 (C-2), 81.0 (C-3), 77.9 (C-4′), 77.7 (C-4), 75.09 (2C, CH₂Ph), 75.06 (C-5′), 74.95 (CH₂Ph), 74.8 (CH₂Ph), 73.6 (C-2′), 73.55 (CH₂Ph), 73.46 (CH₂Ph), 69.8 (C-5), 68.9 (C-6 or C-6′), 68.6 (C-6 or C-6′), 55.2 (OCH₃) ppm; HRMS for [M+Na]⁺: 1023.4296 calcd; found: 1023.4337. The above described data is consistent with those previously described. Mydock and Demchenko Org. Lett. 2008, 10, 11, 2103-2106, DOI: 10.1021/01800345j).

### Example 7

This Example describes the manual and the fully automated synthesis of methyl 2,3,5,6-tetra-O-benzoyl-β-D-galactofuranosyl-(1->3)-2,4,6-tri-O-benzyl-α-D-glucopyranoside (17) (FIG. 10).

Manual. The following four solutions were prepared separately using dry dichloromethane at room temperature. 59 mg of donor 7 (1.3 eq) in 0.9 mL, 30 mg of acceptor 16 (Shrestha, G) (1.0 eq) in 0.9 mL, 25.6 mg of BDMPH (1.4 eq) in 0.9 mL, and 4.5 μL of TBDMSOTf (0.3 eq) n 0.9 mL. In a 10 mL round bottom flask at room temperature, solution of donor, acceptor and BDMPH were added. After that, the resultant mixture was cooled to −20 °C and TBDMSOTf solution was added slowly. The reaction mixture was stirred under argon for 1 h at −0 °C and the whole reaction was loaded into the chromatographic column. 48.5 mg (71%) of pure title compound 17 was isolated. Analytical data coincided with the automated procedure described below.

Automated sequence. All reagent solutions were prepared the same day of the experiment and loaded into the autosampler tray along with Toluene. The reactor was previously cleaned with acetone and dried with hot air. The following four solutions were prepared separately using dry dichloromethane at room temperature. 78.4 mg of donor 7 in 0.9 mL, 40.0 mg of acceptor 16 in 0.9 mL, 34.4 mg of BDMPH in 0.9 mL, and 3.0 μL of TBDMSOTf in 0.9 mL. The execution of the sequence programmed for this reaction gave a set of fractions collected from the fraction collector. Once evaporated, combined fractions yielded 65 mg (72%) of product 17 in pure form. R_{f}=0.50 (DCM/ethyl acetate, 20:1, v/v). [α]_{D}²⁵+25.0 (c=1, CHCl₃). ¹H-NMR (400 MHz, CDCl₃): δ 8.04-7.99 (d, 4H, aromatic), 7.93-7.91 (d, 2H, aromatic), 7.79-7.77 (d, 2H, aromatic), 7.59-7.57 (d, 1H, aromatic), 7.50-7.40 (d, 6H, aromatic), 7.33-7.22 (d, 8H, aromatic), 7.20-7.09 (d, 10H, aromatic), 7.01-6.99 (m, 2H, aromatic), 5.93 (s, 1H, H-1′), 5.83 (m, 1H, J_{5′,6a′}=8.4, J_{5′,6b′}=J_{4′,5′}=3.0 Hz, H-5′), 5.59 (d, 1H, J_{2′,3′}=2.8 Hz, H-2′), 5.52 (dd, 1H, J_{3′,4′}=5.2, J_{2′,3′}=2.8 Hz, H-3′), 4.83 (d, 1H, ²J=11.5 Hz, CHPh), 4.74 (d, 1H, ²J=12.0 Hz, CHPh), 4.63 (d, 1H, J_{1,2}=3.6 Hz, H-1), 4.60 (d, 1H, ²J=12.1 Hz, CHPh), 4.57-4.49 (m, 4H, 2× CHPh, H-4′, H-6a′), 4.45 (d, 1H, ²J=12.1 Hz, CHPh), 4.32 (dd, 1H, J_{2,3}=J_{3,4}=10.2 Hz, H-3), 4.32-4.29 (m, 1H, H-6b′), 3.78 (m, 1H, J_{4,5}=10.2, J_{5,6b}=3.4, J_{5,6a}=2.8 Hz, H-5), 3.69 (dd, 1H, J_{6a,6b}=11.3 Hz, J_{5,6b}=2.8 Hz, H-6a), 3.67-6.31 (m, 2H, H-2, H-6b), 3.62 (dd, 1H, J_{3,4}=J_{4,5}=10.2 Hz, H-4), 3.36 (s, 3H, OCH₃) ppm; ¹³C NMR (101 MHz, CDCl₃): δ 166.0, 165.6, 165.5, 165.3 (COPh×4), 138.0, 137.9, 137.8, 133.4, 133.1, 133.0, 132.8, 129.94, 129.89 129.72, 129.66, 129.2, 129.0, 128.7, 128.43, 128.39, 128.35, 128.30, 128.26, 128.23, 128.20, 128.1, 127.83, 127.81, 127.6, 127.4, 126.6, 106.3 (C-1′), 97.7 (C-1), 82.1 (C-2′), 81.8 (C-4′), 80.7 (C-2), 78.3 (C-3′), 76.2, 76.1 (2C, C-3, C-4), 74.3 (CH₂Ph), 73.4 (CH₂Ph), 73.2 (CH₂Ph), 70.2 (C-5′), 70.1 (C-5), 68.5 (C-6), 64.3 (C-6′), 55.2 (OCH₃) ppm; HRMS for [M+Na]⁺: calcd 1065.3673; found: 1065.3640.

### Example 8

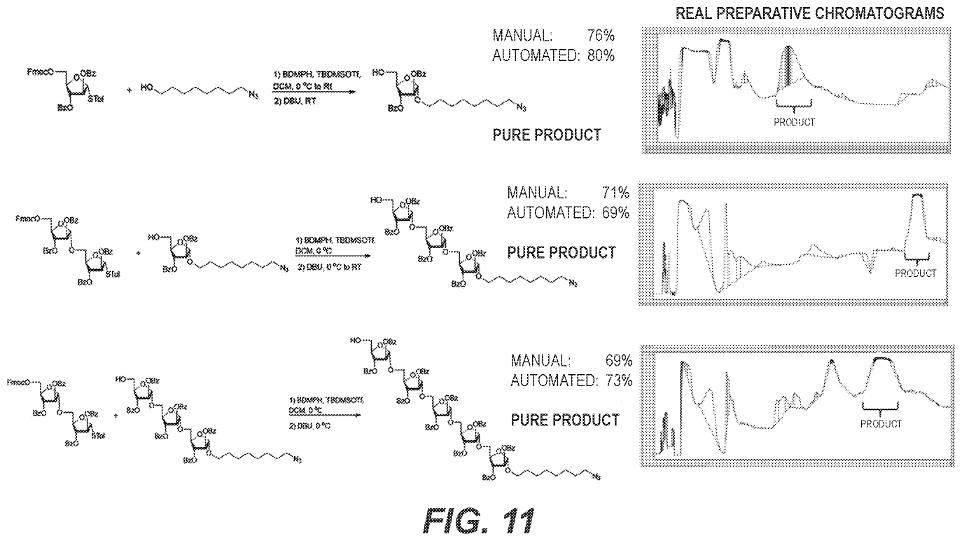

This Example describes the manual and the fully automated synthesis of 8-azidooctyl 2,3-di-O-benzoyl-α-D-arabinofuranoside (20) (FIG. 11).

Observations. Unlike the trichloroacetimidate method, and similar to NIS-promoted methods, it was noted that BDMPH as well as byproducts “NH-hydantoin” had better solubility in DCM. This physical property was relevant to the automation set up, where reagents were delivered in solution through the autosampler. All glycosylation were carried out with dry DCM but with the absence of molecular sieves. The promoter was stable in solid state. But decomposed slowly when it was dissolved in chlorinated solvent like CDCl₃ at room temperature (with no light protection).

Manual: The following four solutions were prepared separately using dry dichloromethane at room temperature. 100 mg of donor 18 in 0.85 mL, 55 μL of 8-azido-1-octanol 19 in 0.85 mL, 87 mg of BDMPH in 0.85 mL, and 20 μL of TBDMSOTf in 0.85 mL, and 65 μL of DBU in 0.85 mL. In a 10 mL round bottom flask at room temperature, solution of donor, acceptor and BDMPH were added. After that, the resultant mixture was cooled to 0 °C under Argon, and TBDMSOTf solution was added slowly. The reaction mixture was allowed to warm to RT and stirred under argon for 25 min, when a TLC showed complete consumption of the donor. After that, DBU solution was added and reaction was allowed to stir for 1.5 h. Then, the whole reaction was loaded into the chromatographic column, which was previously assembled in hexane. 57 mg (76%) of glycoconjugate 20 was isolated. Analytical data coincided with the automated procedure described below.

Automated sequence: All reagent solutions were prepared the same day of the experiment and loaded into the autosampler tray along with Toluene vial. The reactor was previously cleaned with acetone and dried with hot air. The following four solutions were prepared separately using dry dichloromethane at room temperature. 75 mg of donor 18 in 0.9 mL, 44 μL of 8-azido-1-octanol 19 in 0.9 mL, 65 mg of BDMPH in 0.9 mL, 15 μL of TBDMSOTf in 0.9 mL, and 180 μL of DBU in 0.9 mL. The execution of the sequence programmed for this reaction gave a chromatogram pasted below. A set of fractions under the same peak were collected from the fraction collector. Once evaporated, combined fractions yielded 45 mg (80%) of product 20 as a syrup in pure form. R_{f}=0.30 (hexane/ethyl acetate 3.5:1, v/v). ¹H NMR (400 MHz, CDCl₃): δ 8.09-8.04 (m, 4H, aromatic), 7.62-7.57 (m, 2H, aromatic), 7.48-7.44 (m, 4H, aromatic), 5.53 (d, 1H, J=1.4 Hz, H-2), 5.42 (dd, 1H, J=4.1, 1.4 Hz, H-3), 5.23 (s, 1H, H-1), 4.31 (q, 1H, J=4.1 Hz, H-4), 4.04-3.96 (m, 2H, H-5a, H-5b), 3.79-3.73 (m, 1H, OCH₂), 3.55-3.50 (m, 1H, OCH₂), 3.24-3.20 (t, 2H, CH₂N₃), 2.32 (s, 1H, OH), 1.68-1.52 (m, 4H, aliphatic), 1.42-1.26 (m, 8H, aliphatic) ppm. ¹³C NMR (101 MHz, CDCl₃): δ 166.2 (COPh), 165.3 (COPh), 133.5, 129.9, 129.8, 129.2, 129.1, 128.52, 128.46, 105.5 (C-1), 83.6 (C-4), 81.7 (C-2), 77.9 (C-3), 67.4 (OCH₂), 62.4 (C-5), 51.4 (CH₂N₃), 29.5, 29.2, 29.1, 28.8, 26.6, 26.0 (6C, CH₂, alifatic) ppm; HRMS for [M+Na]⁺: calcd 534.2216; found: 534.2171. The described data coincided with previously described (Joe et al. J Am Chem Soc 2007, DOI: 10.1021/ja072892+).

### Example 9

This Example describes the manual and the fully automated synthesis of 8-azidooctyl 2,3-di-O-benzoyl-α-D-arabinofuranosyl-(1->5)-2,3-di-O-benzoyl-α-D-arabinofuranosyl-(1->5)-2,3-di-O-benzoyl-α-D-arabinofuranoside (22) (FIG. 11)

Manual: The following four solutions were prepared separately using dry dichloromethane at room temperature. 54 mg of donor 21 in 0.9 mL, 22 mg of acceptor 20 in 0.9 mL, 19 mg of BDMPH in 0.9 mL, 5 μL of TBDMSOTf in 0.9 mL, and 76 μL of DBU in 0.9 mL. In a 10 mL round bottom flask at room temperature, solution of donor, and acceptor was added. After that, the resultant mixture was cooled to 0 °C, then BDMPH and TBDMSOTf solution was added slowly. The reaction mixture was allowed to be stirred under argon for 35 min, when a TLC showed complete consumption of the donor. After that, DBU solution was added and reaction was stirred at 0 °C for 30 minutes. Then, the whole reaction was loaded into the chromatographic column, which was previously assembled in hexane. 43 mg (71%) of conjugated trisaccharide 22 was isolated. Analytical data coincided with the automated procedure described below

Automated sequence: All reagent solutions were prepared the same day of the experiment and loaded into the autosampler tray along with Toluene. The reactor was previously cleaned with acetone and dried with hot air. The following four solutions were prepared separately using dry dichloromethane at room temperature. 45 mg of donor 21 in 0.9 mL, 18 mg of acceptor 20 in 0.9 mL, 15 mg of BDMPH in 0.9 mL, 4 μL of TBDMSOTf in 0.9 mL, and 62 μL of DBU in 0.9 mL. The execution of the sequence programmed for this reaction gave a set of fractions collected from the fraction collector. Once evaporated, combined fractions yielded 29 mg (69%) of the title product 22 as a foam in pure form. R_{f}=0.33 (hexane/ethyl acetate 2:1, v/v). ¹H NMR (700 MHz, CDCl₃): δ 8.05-8.01 (m, 8H, aromatic), 7.91-7.89 (m, 4H, aromatic), 7.59-7.25 (m, 18H, aromatic), 5.65 (d, 1H, J=1.3 Hz, H-2′), 5.63 (dd, 1H, J=4.0, 1.3 Hz, H-3″), 5.63-5.62 (m, 2H, H-2′, H-3′), 5.50 (d, 1H, J=1.4 Hz, H-2), 5.42 (d, 1H, J=1.4 Hz, H-3), 5.42-5.41 (m, 2H, H-1′, H-1″), 5.21 (s, 1H, H-1), 4.63-4.62 (m, 1H, H-4″), 4.47-4.45 (m, 1H, H-4), 4.42-4.41 (m, 1H, H-4′), 4.19 (dd, 1H, J=11.2, 4.5 Hz, H-5a′), 4.18 (dd, 1H, J=11.4, 4.3 Hz, H-5a″), 4.00 (dd, 1H, J=12.2, 3.9 Hz, H-5a), 3.97 (dd, 1H, J=11.3, 3.1 Hz, H-5b″), 3.96-3.93 (m, 1H, H-5b), 3.92 (dd, 1H, J=11.2, 2.9 Hz, H-5b′), 3.75-3.74 (m, 1H, OCHₐ), 3.50-3.49 (m, 1H, OCH_{b}), 3.21 (t, 2H, J=7.0 Hz, CH₂N₃), 2.3 (bs, 1H, OH), 1.64-1.26 (m, 12H, aliphatic) ppm. ¹³C NMR (101 MHz, CDCl₃): δ 166.1 (COPh), 165.71 (COPh), 165.70 (COPh), 165.5 (COPh), 165.2 (COPh), 165.1 (COPh), 155.2, 133.9, 133.5, 133.45, 133.42, 133.3, 133.2, 129.9, 129.83, 129.79, 129.3, 129.24, 129.18, 129.1, 129.1, 129.07, 129.0, 128.9, 128.5, 128.4, 128.37, 128.32, 128.27, 126.0, 105.84 (C-1′ or C-1″), 105.8 (C-1; or C-1″), 105.6 (C-1), 83.7 (C-4), 82.0 (C-4″), 81.9 (C-4′), 81.8 (C-2), 81.7 (C-2′ or C-2″), 81.6 (C-2′ or C-2″), 77.7 (C-3), 77.2 (C-3′, C-3″), 67.4 (OCH₂), 66.1 (C-5′ or C-5″), 66.0 (C-5′ or C-5″), 62.3 (C-5), 51.4 (CH₂N₃), 29.7, 29.5, 29.3, 29.1, 26.7, 26.1 (6C, CH₂, aliphatic) ppm; HRMS for [M+Na]⁺: calcd 1214.4110; found: 1214.4105. The described data coincided with the previously described (Joe et al. J Am Chem Soc 2007, DOI: 10.1021/ja072892+).

### Example 10

This Example describes the manual and the fully automated synthesis of 8-azidooctyl 2,3-di-O-benzoyl-α-D-arabinofuranosyl-(1->5)-2,3-di-O-benzoyl-α-D-arabinofuranosyl-(1->5)-2,3-di-O-benzoyl-α-D-arabinofuranosyl-(1->5)-2,3-di-O-benzoyl-α-D-arabinofuranosyl-(1->5)-2,3-di-O-benzoyl-α-D-arabinofuranoside (23) (FIG. 11)

Manual: The following four solutions were prepared separately using dry dichloromethane at room temperature. 20.0 mg of donor 21 in 0.5 mL, 18.5 mg of acceptor 22 in 0.5 mL, 7.0 mg of BDMPH in 0.5 mL, 2.0 μL of TBDMSOTf in 0.5 mL, and 28 μL of DBU in 0.5 mL. In a 10 mL round bottom flask at room temperature, solution of donor, acceptor and BDMPH were added. After 1 h of stirring under at 0 °C, a TLC showed total consumption of the donor and acceptor. After that, the whole reaction was loaded into the chromatographic column, which was previously assembled in hexane. 20.1 mg (69%) of conjugated pentasaccharide 23 was isolated. Analytical data coincided with the automated procedure described below

Automated sequence: All reagent solutions were prepared the same day of the experiment and loaded into the autosampler tray along with Toluene. The reactor was previously cleaned with acetone and dried with hot air. The following four solutions were prepared separately using dry dichloromethane at room temperature. 35 mg of donor 21 in 0.9 mL, 24 mg of acceptor 22 in 0.9 mL, 15 mg of BDMPH in 0.9 mL, 3.5 μL of TBDMSOTf in 0.9 mL, and 50 μL of DBU in 0.9 mL. The execution of the sequence programmed for this reaction gave a set of fractions collected from the fraction collector. Once evaporated, combined fractions yielded 27.5 mg (73%) of the title product 23 in pure form. R_{f}=0.32 (hexane/ethyl acetate 3:2, v/v). ¹H NMR (700 MHz, CDCl₃): δ 8.04-7.85 (m, 19H, aromatic), 7.58-7.45 (m, 23H, aromatic), 7.37-7.21 (m, 8H, aromatic), 5.49 (d, 1H, J=1.4 Hz, H-2 araf), 5.40-5.38 (m, 5H, H-3, H-1, H-1, H-1, H-1 araf), 5.20 (s, 1H, H-1 araf), 4.60-4.56 (m, 3H, H-4 araf), 4.45-4.43 (m, 1H, H-4 araf), 4.41-4.40 (m, 1H, H-4 araf), 4.19-4.15 (m, 6H, H-5a, H-5b araf), 3.92-3.89 (m, 6H, H-5a, H-5b araf), 3.73 (m, 1H, OCH₂), 3.48 (m, 1H, OCH₂), 3.21 (t, 2H, J=6.7 Hz, CH₂N₃), 2.25 (m, 1H, OH), 1.68-1.52 (m, 4H, aliphatic), 1.39-1.26 (m, 8H, alifatic) ppm. ¹³C NMR (101 MHz, CDCl₃): δ 166.1 (COPh), 165.66 (2C, COPh), 165.64 (COPh), 165.62 (COPh), 165.4 (COPh), 165.14 (COPh), 165.12 (COPh), 165.10 (COPh), 165.09 (COPh), 133.5, 133.4, 133.3, 133.2, 129.89, 129.87, 129.83, 129.82, 129.79, 129.77, 129.36, 129.26, 129.18, 129.13, 129.06, 129.01, 128.97, 128.51, 128.49, 128.43, 128.31, 128.25, 105.87 (2C, C-1), 105.84 (C-1), 105.81 (C-1), 105.6 (C-1), 83.6, 82.10, 82.08, 82.0, 81.9, 81.8, 81.7, 81.6, 81.55, 81.53, 77.7, 77.3, 77.29, 77.21, 77.03, 67.3 (OCH₂), 66.1, 66.0, 65.8, 65.8, 62.3, 51.4 (CH₂N₃), 29.5, 29.3, 29.1, 28.8, 26.7, 26.1 (6C, CH₂, aliphatic) ppm; HRMS for [M+Na]⁺: calcd 1895.6037; found: 1895.6017. The described data coincided with previously described (Joe et al. J Am Chem Soc 2007, DOI: 10.1021/ja072892+).

### Example 11

This Example describes how the improved system works in application to protecting group manipulations, and first repeated picoloylation to gain the first point of comparison.

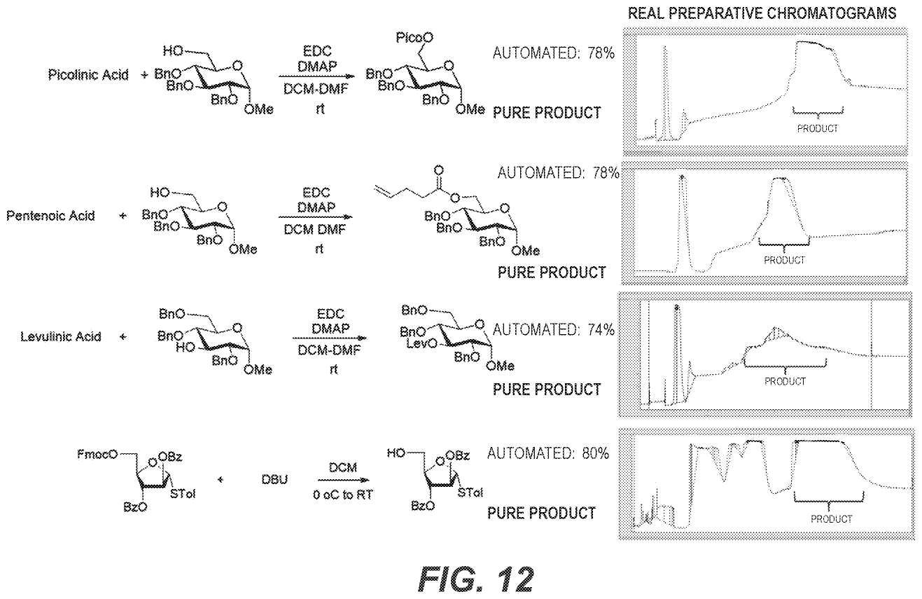

Full automation of the reaction-purification sequence was achieved with the single press of the button. All sequences started with the washing cycle to clean all upstream circuits followed by delivering all reagents with the autosampler to the reactor. The autosampler was programmed to deliver the following solutions from four vials placed into the autosampler tray to achieve the reagent ratio listed in Table 1 (entry 1). Vial 1 contained 6-OH derivative 2 in DCM; Vial 2 contained picolinic acid in DCM; Vial 3 and 4, each contained dimethylaminopyridine (DMAP) and ethylenediaminocarbodiimide (EDC) in DMF-DCM (1/8, v/v). The appropriate sequence of operations was loaded (see general experimental protocol below), and the program was started. Upon completion of the reaction (2 h at rt), subsequent sequence of operations included transfer of the reaction mixture from the reactor to the column, chromatographic separation, and automated collection of purified compounds enabled by the UV detector and fraction collector interface. The post-automation collection and concentration of the respective fractions afforded 6-Pico derivative 24 in 78% yield (FIG. 12). For comparison, the previous automated synthesis was achieved in 16 h and gave compound 2 in 74% yield (see, Kashiwagi et al., Chem. Eur J. 30 2024 Aug. 1; 30 (43): e202401214. doi: 10.1002/chem.202401214. Epub 2024 May 29). The difference in the reaction yield is negligible, but the shorter reaction time could be due the presence of DMF, used to increase the solubility of EDC, which was absent in the previous synthesis.

TABLE 1
Automated synthesis of building blocks via introduction/removal
of common protecting and leaving groups.

| | | | Time | | | |
|---|---|---|---|---|---|---|
| Entry | Substrate | Reagents | Temp | Solvent | Product | Yield |
| 1 | 2 | Picolinic acid (1.5 equiv) | 120 | DCM- | 24 | 78% |
| | | EDC (1.6 equiv), DMAP | min | DMF | | |
| | | (0.5 equiv) | RT | | | |
| 2 | 2 | Pentenoic acid (1.6 equiv), | 180 | DCM- | 25 | 78% |
| | | EDC (1.9 equiv), | min | DMF | | |
| | | DMAP (0.5 equiv) | RT | | | |
| 3 | 16 | Levulinic acid (2.5 equiv) | 190 | DCM- | 26 | 74% |
| | | EDC (2.5 equiv) | min | DMF | | |
| | | DMAP (0.5 equiv) | RT | | | |
| 4 | 18 | DBU (2.4 equiv) | 20 min | DCM | 27 | 80% |
| | | | 0 °C | | | |
| 5 | 28 | DBU (0.4 equiv) | 20 min | DCM | 29 | 81% |
| | | | 0 °C | | | |
| 6 | 30 | Cl₂CCN (2.7 equiv) | 60 min | DCM | 31 | 78% |
| | | DBU (0.4 equiv) | RT | | | |
| 7 | 32 | Cl₂CCN (2.7 equiv) | 5 min | DCM | 33 | 89% |
| | | DBU (0.4 equiv) | 0 °C | | | |

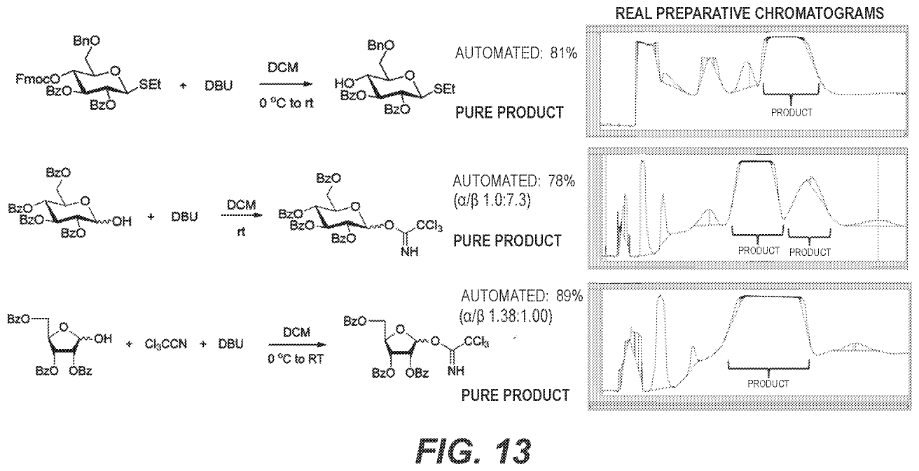

Other esterifications were then investigated to introduce pent-4-enoyl and levulinoyl protecting groups. Esterification of both the primary or secondary hydroxyl in substrates 2 or 16 in the presence of EDC and DMAP, produced the respective compounds 6-Pent 25 or 3-Lev 26 in commendable yields (74-78%, Table 1, entries 2 and 3) (FIG. 12). Deprotection reactions were then investigated, and for this purpose the Fmoc protecting group that is commonly used for temporary protecting of hydroxyls in both manual and automated glycan synthesis was chosen. Removal of Fmoc from primary or secondary positions in substrates 18 or 28 in the presence of DBU was smooth and efficient. The respective products 27 and 29 were obtained in 20 min at 0 °C in 80-81% yield (Table 1, entries 4 and 5) (FIGS. 12 and 13).

Compounds 27 and 29 have a single hydroxyl group and therefore can be used as glycosyl acceptors for couplings with highly reactive glycosyl donors, such as glycosyl trichloroacetimidates. These glycosyl donors found a very broad application in glycosylation and glycan synthesis. To showcase how well the experimental set-up works in application to the synthesis of these common glycosyl donors, pyranose and furanose hemiacetals 30 and 32 were chosen, which reacted with trichloroacetonitrile in the presence of 1,8-diazabicyclo[5.4.0]undec-7-ene (DBU) to afford the corresponding trichloroacetimidate donors 31 and 33 in high yields of 78-89% (Table 1, entries 6 and 7) (FIG. 13).

General methods for examples. DCM used for reactions was distilled from CaH₂ and stored over 3 Å Molecular sieves. Anhydrous DMF was used as it is. ACS grade solvents used for chromatographic separation were used without purification. Chromatographic separations were carried out with Biotage Sfar 10-25 g cartridges packed using low particle size Silica gel SILICAFLASH® R60, 20-45 μm. Compounds collected by the fraction collector were examined by TLC under UV light and by charring with 10% sulfuric acid in methanol. Those fractions enriched by pure product were combined, concentrated, and analyzed. Solvents were removed under reduced pressure at <40 °C. Optical rotations were measured using a Jasco polarimeter. ¹H NMR spectra were recorded in CDCl₃ at 400 or 700 MHz. ¹³C NMR spectra were recorded in CDCl₃ at 101 or 176 MHz. The ¹H NMR chemical shifts are given relative using internal tetramethyl silane (TMS, δ_{H}=0 ppm) standard for solutions in CDCl₃ and HDO signal (δ_{HOD}=4.8 ppm) in case of samples prepared in D₂O. The ¹³C NMR chemical shifts are referenced to the central signal of CDCl₃ (δ_{C}=77.00 ppm) for solutions in CDCl₃. Compound purity or compound ratios were accessed or calculated by comparing the integration intensities of the relevant signals in their ¹H NMR spectra. Accurate mass spectrometry determinations (HRMS) were performed using Agilent 6230 LCMS ESI TOF mass spectrometer.

The general protocol followed for automated reactions was: 1) Addition of molecular sieve beads to the DCM solvent bottle that feeds the quaternary pump 1. 2) Flushing the autosampler and delivery of the capillary tubing with toluene and dry DCM. 3) Washing the reactor with acetone and dry DCM. All the automated syntheses were carried out with the same HPLC set up configured with installed modules. HPLC set up was assisted by external components: a reactor, a magnetic stirrer, a peristaltic pump, a stepper motor and its controller, relays, sensors, a PLC, two solenoid valves, and a preparative medium pressure preparative column. Each sequence followed a specific “Method” written to accomplish a specific aim. Each method was “a program” in which each instrument of the HPLC followed the commands synchronically from ChemStation software. When a method started, all instruments (modules), including internal and external components, worked coordinated in the same timeline. The external components can work independently but are commanded by ChemStation through activation of gate D of the multichannel gradient valve through the quaternary Pumps. Once the reagents were loaded to the synthesizer, the sequence of reactions was downloaded to the software.

Fully automated synthesis of methyl 2,3,4-tri-O-benzyl-6-O-picoloyl-α-D-glucopyranoside (24, FIG. 12). Four vials containing the following solutions were placed into the autosampler tray. Vial 1: methyl 2,3,4-tri-O-benzyl-1-α-D-glucopyranoside (compound 1, 60.0 mg, 0.129 mmol) in DCM (0.9 mL). Vial 2: picolinic acid (24.0 mg, 0.195 mmol) in DCM (0.9 mL). Vial 3 and 4, each containing: DMAP (4.0 mg, 0.033 mmol) and EDC (16 mg, 0.103 mmol) in DMF-DCM (0.9 mL, 1/8, v/v). The sequence of operations was loaded (see general protocol), and the program was started. After 120 minutes of reaction at room temperature followed by purification, the downstream process gave a set of fractions, which were collected and concentrated under reduced pressure and dried in vacuo to afford the title compound as a clear syrup (57.0 mg, 0.100 mmol, 78%). Analytical data for compound 3 were in agreement with those reported in Geringer et al. (J. Org. Chem. 87 (2022): 9887-9895).

### Example 12

Fully automated synthesis of methyl 2,3,4-tri-O-benzyl-6-O-(pent-4-enoyl)-α-D-glucopyranoside (25, FIG. 12). Four vials containing the following solutions were placed into the autosampler tray. Vial 1: compound 1 (110.0 mg, 0.237 mmol) in DCM (0.9 mL). Vial 2: pent-4-enoic acid (34 μL, 0.331 mmol) in DCM (0.9 mL). Vial 3 and 4, each containing: DMAP (7.3 mg, 0.060 mmol) and EDC (34.2 mg, 0.220 mmol) in DMF-DCM (0.9 mL, 2/7, v/v). The sequence of operations was loaded (see general protocol), and the program was started. After 180 minutes of reaction at room temperature followed by purification, the downstream process gave a set of fractions, which were collected and concentrated under reduced pressure and dried in vacuo to afford the title compound as a clear syrup (101.0 mg, 0.185 mmol, 78%). Analytical data for compound 3 were in agreement with those reported in Fujikawa et al. (Chem. Commun. (2011) 10602-10604).

### Example 13

Fully automated synthesis of methyl 2,4,6-tri-O-benzyl-3-O-levulinoyl-α-D-glucopyranoside (compound 26, FIG. 12). Four vials containing the following solutions were placed into the autosampler tray. Vial 1: methyl 2,4,6-tri-O-benzyl-3-O-levulinoyl-α-D-glucopyranoside (compound 4, 110.0 mg, 0.237 mmol) in DCM (0.9 mL). Vial 2: levulinic acid (60 μL, 0.589 mmol) in DCM (0.9 mL). Vial 3 and 4, each containing: DMAP (7.3 mg, 0.060 mmol) and EDC (46.0 mg, 0.296 mmol) in DMF-DCM (0.9 mL, 2/7, v/v). The appropriate sequence of operations was loaded (see general protocol), and the program was started. After 190 minutes of reaction at room temperature followed by purification, the downstream process gave a set of fractions, which were collected and concentrated under reduced pressure and dried in vacuo to afford the title compound as a clear syrup (98.2 mg, 0.175 mmol, 74%). Analytical data for compound 5: R_{f} 0.25 (hexane/ethyl acetate 5/2, v/v); [α]D24+38.3 (c=1, CHCl₃); ¹H NMR (400 MHz, CDCl₃): δ 7.34-7.23 (m, 13H, aromatic), 7.15-7.13 (m, 2H, aromatic), 5.52 (dd, 1H, J_{3,4}=9.6 Hz, H-3), 4.67 (d, 1H, J_{1,2}=3.4 Hz, H-1), 4.63-4.59 (m, 2H, 2× CHPh), 4.54 (d, 1H, ²J=12.3 Hz, CHPh), 4.47 (d, 1H, ²J=12.1 Hz, CHPh), 4.42 (d, 1H, ²J=11.2 Hz, CHPh), 3.77 (m, 1H, H-5), 3.74-3.35 (m, 3H, H-4, 6a, 6b), 3.49 (dd, 1H, J_{2,3}=9.6 Hz, H-2), 3.35 (s, 3H, OCH₃), 2.65-2.62 (m, 2H, CH₂ lev), 2.48-2.42 (m, 2H, CH₂ lev), 2.12 (s, 3H, COCH₃) ppm; ¹³C NMR (101 MHz, CDCl₃): δ 206.5 (CH₃CO-lev), 171.8 (OCO lev), 138.10, 138.07, 137.8, 128.5, 128.3, 128.1, 128.0, 127.9, 127.8, 127.7, 97.9 (C-1), 77.5 (C-2), 76.2 (C-4), 74.3 (CH₂Ph), 74.1 (C-3), 73.6 (CH₂Ph), 72.9 (CH₂Ph), 68.8 (C-5), 68.3 (C-6), 55.3 (OCH₃), 37.9 (CH₂ lev), 29.9 (COCH₃ lev), 28.1 (CH₂ lev) ppm; ESI HRMS for [M+Na]⁺: calcd for C₃₃H₃₈O₈Na 585.2464; found: 585.2453.

### Example 14

Fully automated synthesis of tolyl 2,3-di-O-benzoyl-1-thio-α-D-arabinofuranoside (compound 27, FIG. 12). Three vials containing the following solutions were placed into the autosampler tray. Vial 1 and 2, each containing: tolyl 2,3-di-O-benzoyl-5-O-fluorenylmethoxycarbonyl-1-thio-α-D-arabinofuranoside (compound 6, 110.0 mg, 0.160 mmol) in DCM (0.9 mL). Vial 3: DBU (115 μL, 0.771 mmol) in DCM (0.8 mL). The appropriate sequence of operations was loaded (see general protocol), and the program was started. After 20 minutes of reaction at 0 °C followed by purification, the downstream process gave a set of fractions, which were collected and concentrated under reduced pressure and dried in vacuo to afford the title compound as a white amorphous solid (119.0 mg, 0.256 mmol, 80%). Analytical data for compound 3 were in agreement with those reported in Joe et al. (J. Am. Chem. Soc. 129 (2007) 9885-9901).

### Example 15

Fully automated synthesis of ethyl 2,3-di-O-benzoyl-6-O-benzyl-1-thio-α-D-glucopyranoside (compound 29, FIG. 13). Three vials containing the following solutions were placed into the autosampler tray. Vial 1 and 2, each containing: ethyl 2,3-di-O-benzoyl-6-O-benzyl-4-O-fluorenylmethoxycarbonyl-1-thio-α-D-glucopyranoside (compound 8, 75.0 mg, 0.100 mmol) in DCM (0.9 mL). Vial 3: DBU (72 μL, 0.482 mmol) in DCM (0.8 mL). The appropriate sequence of operations was loaded (see general protocol), and the program was started. After 20 minutes of reaction at 0 °C followed by purification, the down-stream process gave a set of fractions, which were collected and concentrated under reduced pressure and dried in vacuo to afford the title compound as a white amorphous solid (85.3 mg, 0.163 mmol, 81%). Analytical data for compound 29 were in agreement with those reported in Hällgren et al. (J. Carbohydr. Chem. 12 (1993) 309-333).

### Example 16

Fully automated synthesis of 2,3,4,6-tetra-O-benzoyl-α/β-D-glucopyranosyl trichloroacetimidate (compound 31, FIG. 13). Four vials containing the following solutions were placed into the autosampler tray. Vial 1 and 2, each containing: 2,3,4,6-tetra-O-benzoyl-D-glucopyranose (compound 10, 75.0 mg, 0.175 mmol) in DCM (0.9 mL). Vial 3: tichloroacetonitrile (126 μL, 0.683 mmol) in DCM (0.77 mL). Vial 4: DBU (15 μL, 0.101 mmol) in DCM (0.75 mL). The appropriate sequence of operations was loaded (see general protocol), and the program was started. After 60 min of reaction at rt followed by purification, the downstream process gave a set of fractions, which were collected and concentrated under reduced pressure and dried in vacuo to afford the title compound as a clear syrup (145.0 mg, 0.196 mmol, combined yield 78%, a/β=1/7.3). Analytical data for 31 were in agreement with those reported previously (Zeigler et al. Tetrahedron Asymmetry 2000, 11, 3403-3418).

### Example 17

Fully automated synthesis of 2,3,5-tri-O-benzoyl-D-ribofuranosyl trichloroacetimidate (33, FIG. 13). Four vials containing the following solutions were placed into the autosampler tray. Vial 1 and 2, each containing: 2,3,5-tri-O-benzoyl-D-ribofuranose (compound 12, 40.0 mg, 0.086 mmol) in DCM (0.9 mL). Vial 3: tichloroacetonitrile (87 μL, 0.472 mmol) in DCM (0.81 mL). Vial 4: DBU (10 μL, 0.067 mmol) in DCM (0.89 mL). The appropriate sequence of operations was loaded (see general protocol), and the program was started. After 5 minutes of reaction at 0 °C followed by purification, the downstream process gave a set of fractions, which were collected and concentrated under reduced pressure and dried in vacuo to afford the title compound as a clear syrup (93.0 mg, 0.153 mmol, 89%, α/β=1/3.0). Analytical data for compound 33 were in agreement with those previously reported (Chiu-Machado et al., J. Carbohydr. Chem. 2006, 14, 551-561).

### Example 18

This Example illustrates the fully automated synthesis of an intermediate precursor of an active pharmaceutical ingredient, namely uridine tribenzoate, performed using an embodiment of the present disclosure, via N-glycosylation.

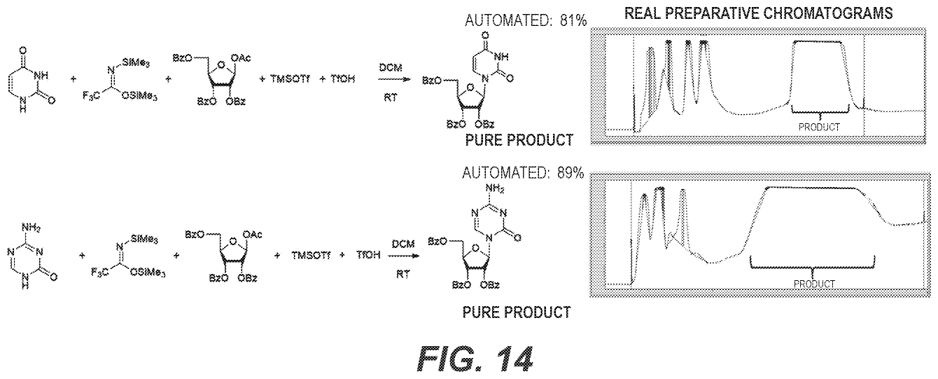

Fully automated synthesis of Uridine tribenzoate (35, FIG. 14). The following four solutions were prepared and disposed onto the autosampler tray. Commercial 1-O-acetyl 2,3,5-tri-O-benzoyl-α-D-ribofuranose (34, 90 mg) in DCM (0.90 mL), N,O-bis(trimethysilyl)trifluoroacetamide (BSTFA) (109 μL) in DCM (0.79 mL), and TMSOTf (40 μL) in DCM (0.86 mL), and TfOH (6.3 μL) in DCM (0.90 mL). Uracil (23 mg) was delivered into the reactor as a solid. After 480 mins of reaction, a downstream process yielded pure nucleoside 35 (81 mg, 81%) as a white solid. R_{f}=0.45 (dichloromethane/ethyl acetate 4:1, v/v). ¹H NMR (400 MHz, CDCl₃) of second fraction: δ 8.63 (d, 1H, J=2.4 Hz, NH), 8.11-7.93 (m, 6H, aromatic), 7.63-7.35 (m, 10H, aromatic), 6.32 (d, 1H, J_{1,2}=5.5 Hz, H-1), 5.89 (dd, 1H, J_{2,3}=5.8, J_{3,4}=4.5 Hz, H-3), 5.75 (dd, 1H, J_{2,3}=5.8, J_{1,2}=5.5 Hz, H-2), 5.61 (dd, 1H, J=8.1, J=2.4 Hz, H-5 uracil), 4.84 (dd, 1H, J_{5a,5b}=12.0, J_{4,5a}=2.6 Hz, H-5a), 4.72-4.71 (m, 1H, H-4), 4.67 (dd, 1H, J_{5a,5b}=12.0 J_{4,5b}=3.9 Hz, H-5b) ppm; ¹³C NMR (101 MHz, CDCl₃): δ 166.0, 165.32, 165.27, 162.4, 149.9, 139.5, 133.83, 133.77, 133.7, 129.9, 129.8, 129.6, 129.2, 128.8, 128.6, 128.3, 103.4 (C-5 uracil), 88.0 (C-1), 80.5 (C-4), 73.7 (C-2), 71.1 (C-3), 63.7 (C-5) ppm. ESI HRMS for [M+K]⁺: calcd 595.1119; found: 595.1127. Analytical data coincided with described (Kashiwagi, et al. Chem Eur J 2024).

### Example 19

This Example illustrates the fully automated synthesis of an intermediate precursor of an active pharmaceutical ingredient, namely azacitidine tribenzoate, performed using an embodiment of the present disclosure, via N-glycosylation.

Fully automated synthesis of Azacitidine tribenzoate (compound 36, FIG. 14). The following four solutions were prepared and disposed of onto the autosampler tray. Commercial 1-O-acetyl 2,3,5-tri-O-benzoyl-α-D-ribofuranose 34 (15, 127 mg) in DCM (0.9 mL), BSTFA (154 μL) in DCM (0.75 mL), TfOH (11 μL) in DCM (0.89 mL), and TMSOTf (56 μL) in DCM (0.84 mL). Azacytosine (32 mg) was delivered into the reactor as a solid. After 960 mins of reaction, a downstream process yielded the title pure nucleoside 36 (125 mg, 89%) as a white solid. R_{f}=0.40 (dichloromethane/ethyl acetate 1:2, v/v). 8.12 (s, 1H, H-6 base), 8.02-7.85 (m, 6H, aromatic), 7.53-7.26 (m, 9H, aromatic), 6.53 (bs, 1H, NH), 6.01 (d, 1H, J_{1,2}=4.1 Hz, H-1), 5.91 (dd, 1H, J_{2,3}=6.0, J_{3,4}=5.9 Hz, H-3), 5.83 (dd, 1H, J_{2,3}=6.0, J_{1,2}=4.1 Hz, H-2), 5.58 (bs, 1H, NH), 4.74 (dd, 1H, J_{5a,5b}=11.5, J_{4,5a}=3.0 Hz, H-5a), 4.71-4.68 (m, 1H, H-4), 4.64 (dd, 1H, J_{5a,5b}=11.5, J_{4,5b}=4.5 Hz, H-5b) ppm; ¹³C NMR (101 MHz, CDCl₃): δ 166.2, 165.9, 165.5, 165.2, 156.4, 152.9, 133.8, 133.7, 133.5, 129.9, 129.8, 129.7, 129.3, 128.6, 128.5, 90.5 (C-1), 80.6 (C-4), 74.8 (C-2), 71.2 (C-3), 63.9 (C-5) ppm. ESI HRMS for [M+K]⁺: calcd 595.1119; found: 595.1127. Analytical data coincided with those described (Kashiwagi, et al. Chem Eur J 2024)

Different from automated carbohydrate synthesizers in flow or automated glycan synthesizers in solid support, the HPLC-A system and methods of the present disclosure alleviate the need to adapt reactions described in literature to an automation method. Also, because the HPLC-A reaction of the present disclosure occurs in a batch reactor and in solution phase, it requires less adaptation to translate from a bench chemistry condition to an automated technology.

The advantage of the HPLC-A system of the present disclosure is that the chemical reaction is performed in solution phase in a batch reactor, while the largest percentage of reactions published in the scientific literature are under same conditions. A reaction described in batch requires a significant amount experimentation and time to be adapted to an automated flow or solid phase manner with no guarantee of reaching same efficiency. Moreover, in the case of automated flow reactors described in the literature, only a very small, restricted percentage of reactions in flow include a telescoped reaction sequence. Additionally, complete automation of reactions in flow is not possible because of the absence of a continuous flow separation method. In the case of automated solid phase glycan synthesis, along with its strategic inconvenience for small glycans, it is well known that non automated (manual) solid phase synthesis usually requires considerable excess of equivalents of coupling building blocks. Through the tandem automated separation of the present invention, the HPLC-A system gains the capability to automate the purification process, thereby achieving an integrated and efficient purification workflow and overcoming a primary limitation historically associated with batch solution-phase reactions as compared to solid-phase methodologies.

What is claimed is:

1. A high performance liquid chromatography equipment-based automation (HPLC-A) system for synthesizing organic molecules, carbohydrate building blocks, nucleosides, oligosaccharides, and drug-like molecules in solution phase under batch conditions comprising:
a high performance liquid chromatography module;
a jacketed reactor;
a chiller;
a chromatography column;
two pumps;
a stir plate;
a programmable logic controller;
two sensors;
a controllable valve; and
a computer.

2. The HPLC-A system of claim 1, wherein the high performance liquid chromatography module comprises: a chromatography column; an autosampler; a detector; a two-position valve; and a fraction collector.

3. The HPLC-A system of claim 1, wherein the computer comprises software configured to control the high performance liquid chromatography module.

4. The HPLC-A system of claim 1, wherein the programmable logic controller comprises software configured to transfer a reaction mixture from the jacketed reactor to the chromatographic column.

5. The HPLC-A system of claim 1, further comprising a separation chromatography column.

6. The HPLC-A system of claim 5, wherein the separation chromatography column is a silica column.

7. The HPLC-A system of claim 1, further comprising an immersible temperature probe system for real time in line temperature measurement of the reaction.

8. The HPLC-A system of claim 1, wherein the two pumps are selected from a peristaltic pump, a controllable stepper motor, and combinations thereof.

9. The HPLC-A system of claim 8, wherein the two pumps are peristaltic pumps, wherein one peristaltic pump is a high-flow peristaltic pump and the one peristaltic pump is a low-flow peristaltic pump.

10. The HPLC-A system of claim 1, wherein the two sensors are selected from a proximity sensor, an optical sensor, and combinations thereof.

11. The HPLC-A system of claim 1, wherein the controllable valve is a solenoid valve.

12. The HPLC-A system of claim 1 further comprising two controllable solenoid valves wherein one controllable solenoid valve is positioned between the reactor and the two way valve, and the other controllable solenoid valve is positioned downstream the column.

13. An automated method for synthesizing and purifying a product selected from organic molecules, carbohydrate building blocks, nucleosides, oligosaccharides, and drug-like molecules, the method comprising:
providing vials containing reactants and reagent solutions and HPLC solvent bottles containing HPLC solvent to the HPLC-A system of claim 1;
initiating an automation sequence; and
collecting the product based on a chromatogram reported by the HPLC-A system.

14. The automated method of claim 13, further comprising concentrating the product.
